(19) United States
(12) Patent Application Publication
(10) Pub. No.: US 2015/0073465 A1
(43) Pub. Date: Mar. 12, 2015

(54) PUNCTURE APPARATUS

(71) Applicant: TERUMO KABUSHIKI KAISHA, Shibuya-ku (JP)

(72) Inventors: Shigeki ARIURA, Ebina-shi (JP); Masakatsu KAWAURA, Sunnyvale, CA (US)

(73) Assignee: TERUMO KABUSHIKI KAISHA, Tokyo (JP)

(21) Appl. No.: 14/547,385

(22) Filed: Nov. 19, 2014

Related U.S. Application Data
(63) Continuation of application No. PCT/JP2013/063998, filed on May 21, 2012.

(30) Foreign Application Priority Data
May 23, 2012 (JP) 2012-118036

(51) Int. Cl. A61B 17/34

(52) U.S. Cl. CPC A61B 17/3468; A61B 17/3403; A61B 17/3494; A61B 2017/00805
USPC 606/185

(57) ABSTRACT

A puncture apparatus is disclosed, which includes a first apparatus, a second apparatus, and an apparatus main body. The first apparatus includes a puncture needle assembly and a support member that supports the puncture needle assembly. The puncture needle assembly includes an outer tube curved into a circular arc shape and an inside structural body composed of a needle body and an implant. Two ultrasonic sensors are placed at the distal portion of the outer tube. The second apparatus can include a vaginal-insertion portion, a urethral-insertion member having a urethral-insertion portion, and an interlock portion that interlocks the vaginal-insertion portion with the urethral-insertion member. Reflective portions that reflect ultrasound are provided on the urethral-insertion portion and the vaginal-insertion portion, respectively.

### CROSS REFERENCES TO RELATED APPLICATIONS

This application is a continuation of International Application No. PCT/JP2013/063998 filed on May 21, 2013, and claims priority to Japanese Application No. 2012-118036 filed on May 23, 2012, the entire content of both of which is incorporated herein by reference.

### TECHNICAL FIELD

The present disclosure relates to puncture apparatus.

### BACKGROUND DISCUSSION

If a person suffers from urinary incontinence, for example, stress urinary incontinence, urine leakage is caused by application of abdominal pressure in normal exercise or due to laughing, coughing, sneezing, or the like. As a cause of this, for example, loosening of the pelvic floor muscle, which is a muscle that supports the urethra, because of childbirth or the like is cited.

Surgical therapy is effective for treatment of the urinary incontinence. For example, a tape-shaped implant called a “sling” is used. The sling is indwelled inside a body and the urethra is supported by the sling (refer to for example, Japanese Patent Laid-open No. 2010-99499). To indwell the sling inside the body, an operator incises the vagina with a surgical knife and dissects a region between the urethra and the vagina. Then, the operator makes the dissected region communicate with the outside through an obturator foramen of the pelvis to form a puncture hole by using a puncture needle. This formation of the puncture hole by the puncture needle is performed by blind operation based only on the sense of fingers. Then, the sling is indwelled inside the body by using such a puncture hole.

However, the method in which the sling is indwelled by using a conventional medical device such as the surgical knife can involve a defect that the invasion can be relatively great and burdensome on the patient. Furthermore, when the vagina is incised with the surgical knife and a region between the urethra and the vagina is dissected, the dissection can be performed in a wrong direction, and the sling cannot be indwelled at a proper position, such that the urethra can be damaged. The fingertip of the operator can also be damaged. In addition, the puncture route of the puncture needle can deviate from the target route in the formation of the puncture hole by the puncture needle due to the movement of the patient or the like such that the operator may be unaware of this in some cases. This can cause the urethra to be damaged, the sling may not be indwelled at a proper position, and the occurrence of a complication called “erosion” in which the sling breaks the vaginal wall to be exposed to the inside of the vagina after the operation, or the like can occur.

### SUMMARY

In accordance with an exemplary embodiment, a puncture apparatus is disclosed that can prevent the region that must not be punctured from being punctured when living body tissues are punctured by a puncture needle, and can provide a relatively light burden on a patient with relatively high safety of the patient and operator.

In accordance with an exemplary embodiment, a puncture apparatus is disclosed, which can include an insertion portion that is insertable into a living body, a puncture needle that punctures living body tissues near the insertion portion in a state in which the insertion portion is inserted into the living body, and detection means that detects information relating to a positional relationship between a distal portion of the puncture needle and the insertion portion.

In accordance with an exemplary embodiment, in the puncture apparatus of the present disclosure, it can be preferable that the insertion portion is a urethral-insertion portion that is inserted into a urethra and has an elongated shape or a vaginal-insertion portion that is inserted into a vagina and has an elongated shape.

In accordance with an exemplary embodiment, in the puncture apparatus of the present disclosure, it can be preferable that the insertion portion is a urethral-insertion portion that is inserted into a urethra and has an elongated shape, and the puncture apparatus has a restriction unit that is provided on the urethral-insertion portion and restricts the position of the urethral-insertion portion in the longitudinal direction in the urethra.

In accordance with an exemplary embodiment, in the puncture apparatus of the present disclosure, it can be preferable that the detection means has a function to detect each of pieces of information relating to positional relationships between the distal portion of the puncture needle and two different regions of the insertion portion in the longitudinal direction.

In accordance with an exemplary embodiment, in the puncture apparatus of the present disclosure, it can be preferable that the puncture apparatus has two insertion portions as the insertion portion.

In accordance with an exemplary embodiment, in the puncture apparatus of the present disclosure, it can be preferable that one of the two insertion portions is a urethral-insertion portion that is inserted into a urethra and has an elongated shape, and the other is a vaginal-insertion portion that is inserted into a vagina and has an elongated shape.

In accordance with an exemplary embodiment, in the puncture apparatus of the present disclosure, it can be preferable that the detection means has a function to detect each of pieces of information relating to positional relationships between the distal portion of the puncture needle and two different regions of the insertion portion in the longitudinal direction.

In accordance with an exemplary embodiment, in the puncture apparatus of the present disclosure, it can be preferable that the detection means has a function to detect information relating to a positional relationship between one and the other of the two insertion portions.

In accordance with an exemplary embodiment, in the puncture apparatus of the present disclosure, it can be preferable that the puncture apparatus has informing means that informs information based on a detection result of the detection means.

In accordance with an exemplary embodiment, in the puncture apparatus of the present disclosure, it can be preferable that the puncture apparatus has determination means that determines whether or not the separated distance between the one and the other is equal to or larger than a minimum value of an allowable range, and an alarm is made by the informing means if it is determined by the determination means that the separated distance between the one and the other is smaller than the minimum value of the allowable range.

In accordance with an exemplary embodiment, in the puncture apparatus of the present disclosure, it can be preferable that the determination means has a function to determine whether or not the separated distance between the one and the other is equal to or smaller than a maximum value of an allowable range, and an alarm is made by the informing means if it is determined by the determination means that the separated distance between the one and the other is larger than the maximum value of the allowable range.

In accordance with an exemplary embodiment, in the puncture apparatus of the present disclosure, it can be preferable that the puncture apparatus has informing means that informs information based on a detection result of the detection means.

In accordance with an exemplary embodiment, in the puncture apparatus of the present disclosure, it can be preferable that the puncture apparatus has determination means that determines whether or not the separated distance between the distal portion of the puncture needle and the insertion portion is equal to or larger than a minimum value of an allowable range, and an alarm is made by the informing means if it is determined by the determination means that the separated distance between the distal portion of the puncture needle and the insertion portion is smaller than the minimum value of the allowable range.

In accordance with an exemplary embodiment, in the puncture apparatus of the present disclosure, it can be preferable that the puncture apparatus has determination means that determines whether or not the separated distance between the distal portion of the puncture needle and the insertion portion is equal to or smaller than a maximum value of an allowable range, and an alarm is made by the informing means if it is determined by the determination means that the separated distance between the distal portion of the puncture needle and the insertion portion is larger than the maximum value of the allowable range.

In accordance with an exemplary embodiment, in the puncture apparatus of the present disclosure, it can be preferable that the puncture apparatus has informing means that informs information based on a detection result of the detection means, and comparison means that compares one of the separated distances between the distal portion of the puncture needle and the two different regions of the insertion portion in the longitudinal direction with the other, and the informing means informs information based on a comparison result of the comparison means.

In accordance with an exemplary embodiment, in the puncture apparatus of the present disclosure, it can be preferable that the puncture needle is curved into a shape of a circular arc, and the puncture apparatus has a support portion that supports the puncture needle rotationally movably around a center axis of the circular arc, and a guide portion that is provided at a different position from the support portion and is located in a direction toward which a needle tip of the puncture needle moves when the puncture needle rotationally moves.

In accordance with an exemplary embodiment, in the puncture apparatus of the present disclosure, it can be preferable that the puncture needle is formed of a puncture needle assembly that includes an outer tube that is formed of a tubular body curved into a shape of a circular arc and has a one-end opening obtained by opening of one end of the outer tube and a needle body that is freely detachably mounted at the one-end opening and has a rounded, non-sharp needle tip to puncture living body tissues, the puncture needle assembly being capable of taking an assembled state in which the puncture needle assembly is assembled by mounting the needle body at the one-end opening, and the puncture apparatus has a support portion that supports the puncture needle assembly in the assembled state rotationally movably around a center axis of the circular arc of the outer tube, and a guide portion that is provided at a different position from the support portion and is located in a direction toward which the needle tip moves when the puncture needle assembly rotationally moves.

In accordance with an exemplary embodiment, according to the present disclosure, the region that must not be punctured can be prevented from being punctured when living body tissues are punctured by the puncture needle. In addition, the burden on the patient can be relatively light and the safety of the patient can be relatively high. Furthermore, the safety of the operator can also be relatively high.

For example, when the puncture apparatus of the present disclosure is used for treatment of urinary incontinence of a woman, the insertion portion of the puncture apparatus is inserted into a urethra and the puncture needle is rotationally moved to puncture the living body by the puncture needle. At this time, information relating to the positional relationship between the distal portion of the puncture needle and the insertion portion can be detected and thus puncture of the urethra by the puncture needle can be prevented.

Furthermore, in implanting an implant for treatment of urinary incontinence, incision of the vaginal wall is unnecessary and the implant can be implanted by a less-invasive procedure. Moreover, damage to a fingertip to the operator can be prevented.

In accordance with an exemplary embodiment, a puncture apparatus is disclosed comprising: an insertion portion that is insertable into a living body; a puncture needle that punctures living body tissues near the insertion portion in a state in which the insertion portion is inserted into the living body; and detection means that detects information relating to a positional relationship between a distal portion of the puncture needle and the insertion portion.

In accordance with an exemplary embodiment, a puncture apparatus is disclosed comprising: a puncture needle that punctures living body tissues near the insertion portion in a state in which the insertion portion is inserted into the living body, the puncture needle having at least one ultrasonic sensor on a distal portion of the puncture needle; an insertion portion that is insertable into a living body, the insertion portion comprising at least one of a urethral-insertion portion that is inserted into a urethra and has an elongated shape and a vaginal-insertion portion that is inserted into a vagina and has an elongated shape, the insertion portion having at least one reflective portion that reflects ultrasound; and detection means that detects information relating to a positional relationship between the distal portion of the puncture needle and the insertion portion based on the at least one ultrasonic sensor on the puncture needle and the at least one reflective portion on the insertion portion.

In accordance with an exemplary embodiment, a method of preventing a region of a living body from being punctured is disclosed, comprising: inserting an insertion portion into a living body, the insertion portion comprising at least one of a urethral-insertion portion that is inserted into a urethra and has an elongated shape and a vaginal-insertion portion that is inserted into a vagina and has an elongated shape; inserting a puncture needle that punctures living body tissues near the insertion portion in a state in which the insertion portion is inserted into the living body; detecting a positional relationship between a distal portion of the puncture needle and the insertion portion; and informing an operator of the positional relationship between the distal portion of the puncture needle and the insertion portion using an informing means.

### BRIEF DESCRIPTION OF THE DRAWINGS

FIG. 1 is a partial sectional view illustrating a first exemplary embodiment of a puncture apparatus of the present disclosure.

FIGS. 2A-2D are diagrams illustrating the puncture apparatus illustrated in FIG. 1, wherein FIG. 2A is a side view, FIG. 2B is a front view, FIG. 2C is a back view, and FIG. 2D is a sectional view along line A-A in FIG. 2A.

FIG. 3 is a block diagram illustrating the circuit configuration of the puncture apparatus illustrated in FIG. 1.

FIG. 4 is a flowchart illustrating control operation of a control unit of the puncture apparatus illustrated in FIG. 1.

FIG. 5 is a diagram for explaining a step of a procedure when the puncture apparatus illustrated in FIG. 1 is used.

FIG. 6 is a diagram for explaining a step of the procedure when the puncture apparatus illustrated in FIG. 1 is used.

FIG. 7 is a diagram for explaining a step of the procedure when the puncture apparatus illustrated in FIG. 1 is used.

FIG. 8 is a diagram for explaining a step of the procedure when the puncture apparatus illustrated in FIG. 1 is used.

FIG. 9 is a diagram for explaining a step of the procedure when the puncture apparatus illustrated in FIG. 1 is used.

FIG. 10 is a diagram for explaining a step of the procedure when the puncture apparatus illustrated in FIG. 1 is used.

FIG. 11 is a diagram for explaining a step of the procedure when the puncture apparatus illustrated in FIG. 1 is used.

FIG. 12 is a diagram for explaining a step of the procedure when the puncture apparatus illustrated in FIG. 1 is used.

FIG. 13 is a partial sectional view illustrating a second exemplary embodiment of the puncture apparatus of the present disclosure.

FIGS. 14A-14C are diagrams illustrating the puncture apparatus illustrated in FIG. 13.

FIG. 15 is a block diagram illustrating the circuit configuration of the puncture apparatus illustrated in FIG. 13.

FIG. 16 is a flowchart illustrating control operation of a control unit of the puncture apparatus illustrated in FIG. 13.

FIG. 17 is a diagram for explaining a step of a procedure when the puncture apparatus illustrated in FIG. 13 is used.

FIG. 18 is a diagram for explaining a step of the procedure when the puncture apparatus illustrated in FIG. 13 is used.

FIG. 19 is a diagram for explaining a step of the procedure when the puncture apparatus illustrated in FIG. 13 is used.

FIG. 20 is a diagram for explaining a step of the procedure when the puncture apparatus illustrated in FIG. 13 is used.

FIG. 21 is a diagram for explaining a step of the procedure when the puncture apparatus illustrated in FIG. 13 is used.

FIG. 22 is a diagram for explaining a step of the procedure when the puncture apparatus illustrated in FIG. 13 is used.

FIG. 23 is a diagram for explaining a step of the procedure when the puncture apparatus illustrated in FIG. 13 is used.

FIG. 24 is a diagram for explaining a step of the procedure when the puncture apparatus illustrated in FIG. 13 is used.

FIGS. 25A-25C are diagrams illustrating a third exemplary embodiment of the puncture apparatus of the present disclosure, wherein FIG. 25A is a side view, FIG. 25B is a front view, and FIG. 25C is a back view.

FIG. 26 is a block diagram illustrating the circuit configuration of the puncture apparatus illustrated in FIG. 25.

FIG. 27 is a flowchart illustrating control operation of a control unit of the puncture apparatus illustrated in FIG. 25.

FIGS. 28A-28C are partial sectional views illustrating a fourth exemplary embodiment of the puncture apparatus of the present disclosure.

### DETAILED DESCRIPTION

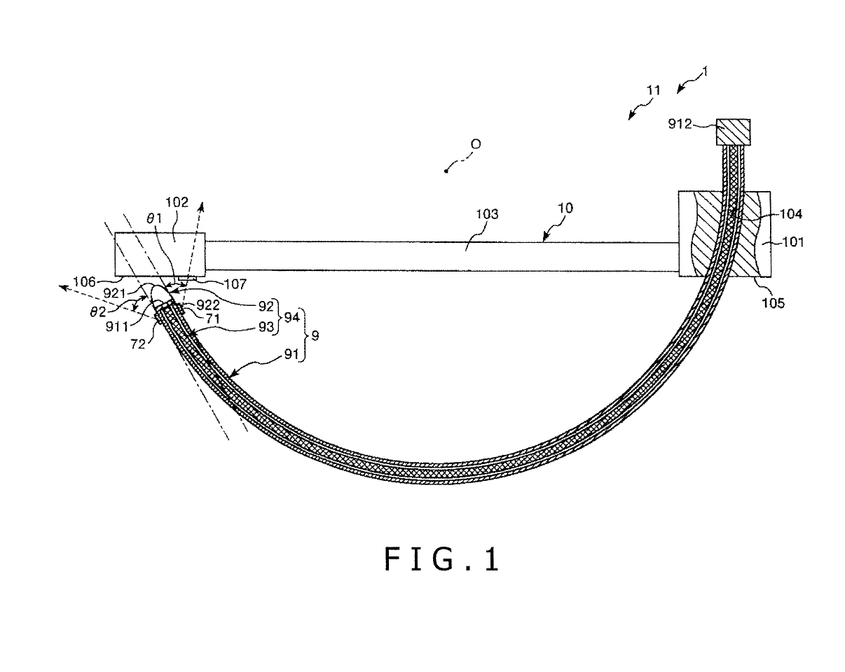

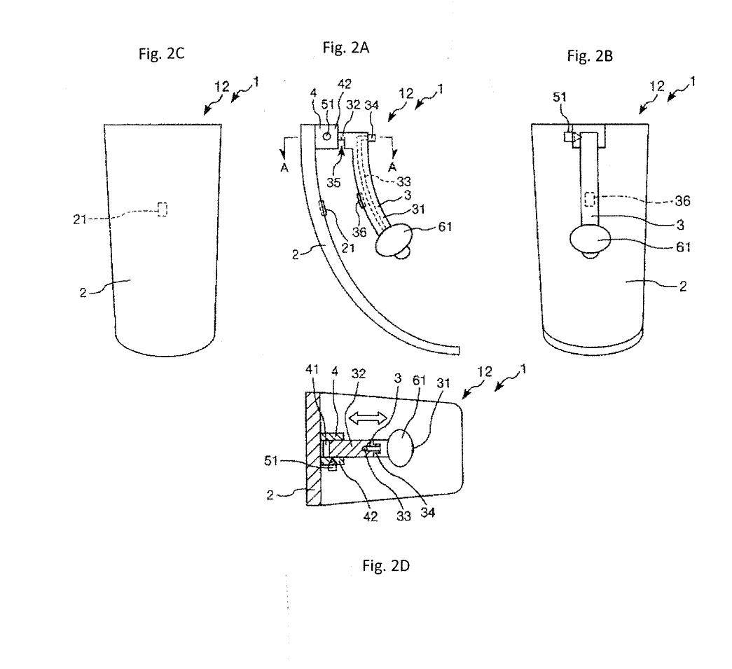

In the following, description will be so made that the left side is defined as the “distal end” and the right side is defined as the “proximal end” along the longitudinal direction of a puncture needle assembly in FIG. 1 and the lower side is defined as the “distal end” and the upper side is defined as the “proximal end” along the longitudinal direction of a vaginal-insertion portion and a urethral-insertion portion in FIGS. 2A, 2B, and 2C.

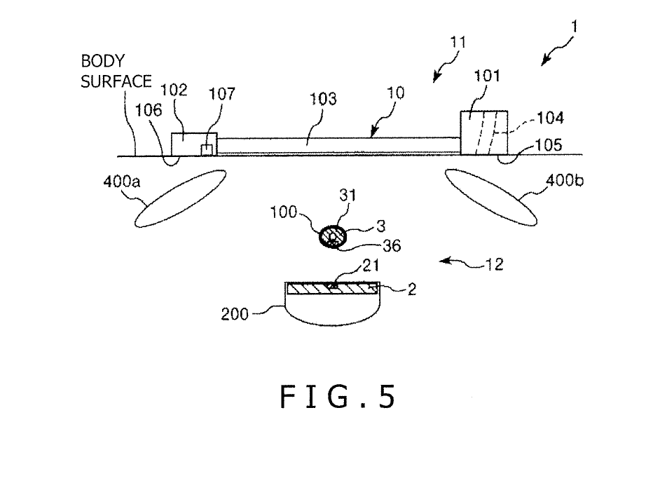

Furthermore, in each of FIGS. 5 to 12, for high visibility, each ultrasonic sensor and each reflective portion are represented with a large size schematically and slant lines in the living body are omitted.

In accordance with an exemplary embodiment, a puncture apparatus 1 as illustrated in these diagrams can be used for treatment of urinary incontinence of a woman, for example, used when an implant (in-vivo indwelling device) for treatment of urinary incontinence is implanted in a living body.

As illustrated in FIGS. 1 to 3, the puncture apparatus 1 includes first apparatus 11, second apparatus 12, and an apparatus main body 13.

As illustrated in FIG. 1, the first apparatus 11 includes a puncture needle assembly 9 and a support member 10 as a puncture needle.

The puncture needle assembly 9 can be curved into a circular arc shape. This puncture needle assembly 9 can include an outer tube 91 formed of a tubular body curved into a circular arc shape and an inside structural body 94 composed of a needle body 92 and an implant 93. Furthermore, the puncture needle assembly 9 can take an assembled state in which the outer tube 91 and the inside structural body 94 are assembled (see FIGS. 1 and 6 to 9) and a decomposed state in which the outer tube 91 and the inside structural body 94 are separated from the assembled state (see FIGS. 10 to 12). Moreover, the puncture needle assembly 9 punctures, in the assembled state, living body tissues near a urethral-insertion portion 31 and a vaginal-insertion portion 2 to be described later, for example, between the urethral-insertion portion 31 and the vaginal-insertion portion 2, in a state in which the urethral-insertion portion 31 and the vaginal-insertion portion 2 are inserted into a urethra and a vagina, respectively.

The outer tube 91 has an one-end opening 911 obtained by opening of one end of the outer tube 91 and has a grasping unit 912 at the other end portion. The needle body 92 can be freely detachably mounted at the one-end opening 911. Furthermore, the grasping unit 912 serves as a portion grasped when the outer tube 91 is drawn out from living body tissues as described later.

In accordance with an exemplary embodiment, the central angle of the outer tube 91 forming a circular arc shape, for example, is about 135 to 210°, and for example, preferable that the central angle is about 150 to 180°.

The needle body 92 can form a mushroom shape and its top portion can serve as a needle tip 921 that can puncture living body tissues. The needle tip 921 may be somewhat rounded so that an operator may be prevented from accidentally puncturing an operator's own finger or the like. Furthermore, a step portion 922 at which the outer diameter steeply changes is formed at the proximal portion of the needle body 92, for example, a portion on the opposite side to the needle tip 921.

The implant 93 is an implantable device for treatment of urinary incontinence of a woman and can be an elongated member interlocked with the proximal portion of the needle body 92. This implant 93 forms a mesh shape and has flexibility. The implant 93 is indwelled in living body tissues together with the needle body 92 and supports the urethra. When the urethra tries to move toward the vaginal wall for example, the implant 93 supports the urethra in such a manner as to draw the urethra in the direction in which the urethra gets farther away from the vaginal wall.

Furthermore, in the assembled state, the needle body 92 is mounted at the one-end opening 911 of the outer tube 91 and the implant 93 is inserted in the outer tube 91. In the decomposed state, the needle body 92 separates from the one-end opening 911 of the outer tube 91 and the implant 93 is withdrawn from the outer tube 91.

The constituent material of the outer tube 91 is not particularly limited. For example, metal materials such as stainless steel, aluminum or aluminum alloy, and titanium or titanium alloy can be used. Furthermore, the constituent material of the needle body 92 and the implant 93 is not particularly limited. For example, various kinds of resin materials having biocompatibility, such as polypropylene, fiber, and so forth can be used.

Furthermore, two ultrasonic sensors (ultrasonic oscillators) 71 and 72 that transmit and receive ultrasound are placed at the distal portion of the puncture needle assembly 9, for example, on the outer circumferential surface of the distal portion of the outer tube 91. The ultrasonic sensor 71 is a component that transmits/receives ultrasound to/from a reflective portion 36 of the urethral-insertion portion 31 to be described later, for example, a component that transmits ultrasound to the reflective portion 36 and receives ultrasound reflected by the reflective portion 36. In addition, the ultrasonic sensor 72 is a component that transmits/receives ultrasound to/from a reflective portion 21 of the vaginal-insertion portion 2 to be described later, for example, a component that transmits ultrasound to the reflective portion 21 and receives ultrasound reflected by the reflective portion 21. These ultrasonic sensors 71 and 72 each have, for example, an ultrasonic oscillator made by forming electrodes on both surfaces of a piezoelectric body composed of PZT (lead zirconate titanate) or the like.

The ultrasonic sensor 71 is disposed on the inner circumferential side of the circular arc as the curved shape of the puncture needle assembly 9 so as to be located on the side of the urethral-insertion portion 31 when living body tissues are punctured by the puncture needle assembly 9.

Furthermore, it can be preferable that the ultrasonic sensor 71 is so placed that an angle θ1 formed by the center line of ultrasound transmitted from the ultrasonic sensor 71 and a tangent to the outer tube 91 at the site where the ultrasonic sensor 71 is placed is, for example, about 15° to 75°, and it can be more preferable that the ultrasonic sensor 71 is so set that the angle θ1 is, for example, about 30° to 60°. In accordance with an exemplary embodiment, θ1 is set to 45° in the configuration illustrated in the diagram.

Meanwhile, the ultrasonic sensor 72 is disposed on the outer circumferential side of the circular arc as the curved shape of the puncture needle assembly 9 so as to be located on the side of the vaginal-insertion portion 2 when living body tissues are punctured by the puncture needle assembly 9.

Furthermore, it can be preferable that the ultrasonic sensor 72 is so placed that an angle 82 formed by the center line of ultrasound transmitted from the ultrasonic sensor 72 and a tangent to the outer tube 91 at the site where the ultrasonic sensor 72 is placed is, for example, about 15° to 75°, and it can be more preferable that the ultrasonic sensor 72 is so set that the angle 82 is, for example, about 30° to 60°. In accordance with an exemplary embodiment, θ2 is set to 45° in the configuration illustrated in the diagram.

In accordance with an exemplary embodiment the number of ultrasonic sensors is not limited to two and may be, for example, one, or three or more.

The support member 10 has a support portion 101, a guide portion 102 disposed at a different position from the support portion 101, for example, disposed separately from the support portion 101, and an interlock portion 103 that interlocks the support portion 101 with the guide portion 102.

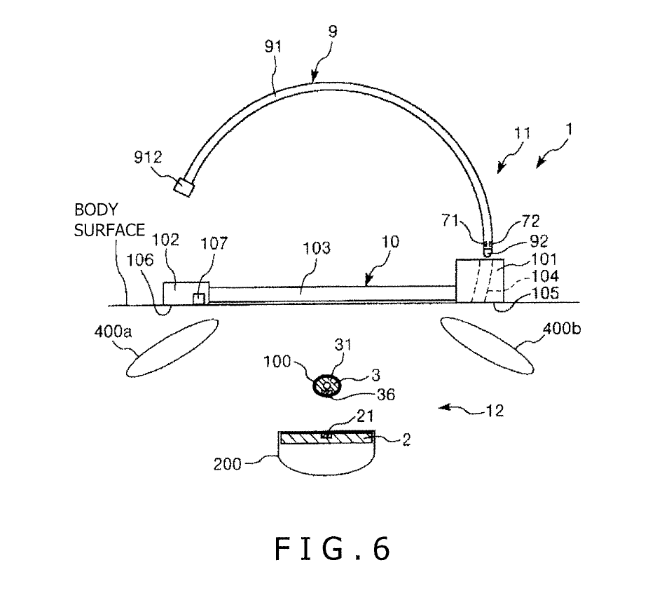

The support portion 101 is a member that is formed of a block body of a cuboid or cube and in which a through-hole 104 penetrating the block body is formed. The puncture needle assembly 9 (outer tube 91) in the assembled state is insertable into the through-hole 104 and the through-hole 104 is curved with a curvature equivalent to that of the outer tube 91 (puncture needle assembly 9). Furthermore, by this through-hole 104, the puncture needle assembly 9 in the assembled state is supported rotationally movably around an axis whose center axis is the center O of the circular arc of the outer tube 91 (see FIGS. 6 to 9).

Furthermore, a surface 105 of the support portion 101 on the lower side in FIG. 1 functions as a put portion put on a body surface. In accordance with an exemplary embodiment, the support portion 101 is not limited to the block body and may have, for example, a shape including a curved surface.

The guide portion 102 is a member that is formed of a block body of a cuboid or cube and is located in the direction toward which the needle tip 921 of the needle body 92 moves when the puncture needle assembly 9 in the assembled state rotationally moves. In the present embodiment, the needle tip 921 of the needle body 92 is supposed to move toward the center portion of the guide portion 102.

A surface 106 of the guide portion 102 on the lower side in FIG. 1, for example, a surface to face the needle tip 921, functions as a put portion put on a body surface. In accordance with an exemplary embodiment, the guide portion 102 is not limited to the block body and may have for example, a shape including a curved surface.

A reflective portion 107 that reflects ultrasound is provided on the surface 106 of the guide portion 102. The position of this reflective portion 107 is not particularly limited. In the present exemplary embodiment, the reflective portion 107 can be disposed at such a position that the center axis of ultrasound transmitted from the ultrasonic sensor 71 penetrates the reflective portion 107 when living body tissues are punctured by the puncture needle assembly 9 and the needle tip 921 of the puncture needle assembly 9 is brought close to the reflective portion 107, for example, at an end portion of the guide portion 102 on the interlock portion side.

Furthermore, the reflective portion 107 is not particularly limited as long as it can reflect ultrasound. For example, as its constituent material, an object obtained by using a metal material such as stainless steel, aluminum or aluminum alloy, or titanium or titanium alloy and forming plural minute concavities and convexities on a surface, and so forth can be used. In addition, if the guide portion 102 is composed of a metal material, plural minute concavities and convexities may be formed on a surface of the guide portion 102 as the reflective portion 107.

The interlock portion 103 is formed of an elongated body and supports the support portion 101 and the guide portion 102 at its both end portions, respectively. Due to this, the support portion 101 is interlocked with the guide portion 102 via the interlock portion 103.

Furthermore, in the puncture apparatus 1, the puncture needle assembly 9 can be operated in a state in which the urethral-insertion portion 31 of the second apparatus 12 to be described later is inserted into a urethra and the vaginal-insertion portion 2 is inserted into a vagina, and the surface 105 of the support portion 101 is put on a body surface and the surface 106 of the guide portion 102 is put on the body surface (hereinafter, this state will be referred to as the “use state”) (see FIGS. 5 to 12). In accordance with an exemplary embodiment, a puncture hole for implanting the implant 93 can be formed in living body tissues by rotationally moving the puncture needle assembly 9 with the center O of the circular arc of the outer tube 91 being the center of the rotary movement.

As illustrated in FIG. 2, the second apparatus 12 includes the vaginal-insertion portion (insertion portion) 2 that is inserted into a vagina and has an elongated shape, a urethral-insertion member 3 having the urethral-insertion portion (insertion portion) 31 that is inserted into a urethra and has an elongated shape, and an interlock portion 4 as interlock means that interlocks the vaginal-insertion portion 2 with the urethral-insertion member 3.

The shape of the vaginal-insertion portion 2 is not particularly limited as long as it is an elongated shape. In the present exemplary embodiment, the vaginal-insertion portion 2 forms a plate shape. Furthermore, the width of the vaginal-insertion portion 2 gradually decreases in its distal direction and the thickness of the vaginal-insertion portion 2 gradually decreases in its distal direction. Furthermore, the distal portion of the vaginal-insertion portion 2 is rounded, which can help enhance the safety of the patient.

Moreover, the vaginal-insertion portion 2 is curved. Due to this, the operative field can be oriented toward the front side of the operator when the vaginal-insertion portion 2 is inserted into a vagina, so that the operative field can be set wide.

The urethral-insertion member 3 has the urethral-insertion portion 31 and a protrusion 32 that is formed at the proximal portion of the urethral-insertion portion 31 and protrudes from the urethral-insertion portion 31 toward the left side in FIG. 2A.

The shape of the urethral-insertion portion 31 is not particularly limited as long as it is an elongated shape. In the present exemplary embodiment, the urethral-insertion portion 31 forms a bar shape. Furthermore, the distal portion of the urethral-insertion portion 31 is rounded, which can help enhance the safety of the patient.

Moreover, the urethral-insertion portion 31 is curved in the same direction as the vaginal-insertion portion 2. The curvature of this urethral-insertion portion 31 is set equal to that of the vaginal-insertion portion 2. Furthermore, the posture of the urethral-insertion portion 31 is so set that the separated distance between the urethral-insertion portion 31 and the vaginal-insertion portion 2 is constant along the longitudinal direction of the urethral-insertion portion 31.

The interlock portion 4 is fixed to the right side, in FIG. 2(a), of the proximal portion of the vaginal-insertion portion 2.

In this interlock portion 4, a bottomed hole 41 is formed along the left-right direction in FIGS. 2A and 2D. In accordance with an exemplary embodiment the right side of the hole 41 in FIGS. 2A and 2D is opened.

In the hole 41, the protrusion 32 of the urethral-insertion member 3 is inserted movably in its longitudinal direction. By the movement of the urethral-insertion member 3 relative to the interlock portion 4 in the longitudinal direction of the protrusion 32, the separated distance 35 between the vaginal-insertion portion 2 and the urethral-insertion portion 31 can be changed. Therefore, adjustment means that adjusts the separated distance 35 between the vaginal-insertion portion 2 and the urethral-insertion portion 31 is configured by the hole 41 of the interlock portion 4 and the protrusion 32 of the urethral-insertion member 3. Because the patient involves individual difference and the separated distance between the vagina and the urethra often differs depending on the patient, the separated distance between the vaginal-insertion portion 2 and the urethral-insertion portion 31 can be appropriately adjusted to fit the patient by the adjustment means.

Furthermore, the second apparatus 12 has a male screw 51, and a female screw portion 42 having a female screw screwed to the male screw 51 is formed in the interlock portion 4.

When the male screw 51 is rotated in a predetermined direction, the distal end of the male screw 51 comes into pressure contact with the protrusion 32 of the urethral-insertion member 3 and the movement of the urethral-insertion member 3 relative to the interlock portion 4 is inhibited. Furthermore, when the male screw 51 is rotated in the opposite direction of the aforementioned direction, the distal end of the male screw 51 gets separated from the protrusion 32 and the movement of the urethral-insertion member 3 relative to the interlock portion 4 is allowed.

In accordance with an exemplary embodiment a locking portion that switches the state between the state in which the urethral-insertion member 3 can move relative to the interlock portion 4 and the state in which the movement of the urethral-insertion member 3 is inhibited is configured by the male screw 51 and the female screw portion 42.

Furthermore, at the distal portion of the urethral-insertion portion 31, an inflatable and deflatable balloon 61 is provided as a restriction unit that restricts the position of the urethral-insertion portion 31 in the longitudinal direction in a urethra. When the second apparatus 12 is used, the balloon 61 is inserted into the bladder of a patient and the balloon 61 catches on the bladder neck in the inflated state. The position of the urethral-insertion portion 31 relative to the bladder and the urethra can thereby be fixed.

Furthermore, a lumen 33 is formed in the urethral-insertion portion 31. The distal end of this lumen 33 is opened in the balloon 61 and the proximal end is opened to a side surface of the proximal portion of the urethral-insertion portion 31. In addition, a port 34 communicating with the proximal end of the lumen 33 can be formed at the proximal portion of the urethral-insertion portion 31.

The balloon 61 is inflated or deflated by connecting a balloon inflation device such as a syringe not illustrated to the port 34 and sending a working fluid supplied from the balloon inflation device into the inside of the balloon 61 via the lumen 33 or drawing the working fluid. In accordance with an exemplary embodiment for example, a liquid such as a physiological saline solution, a gas, or the like can be used as the working fluid for the balloon inflation.

In accordance with an exemplary embodiment, the restriction unit to restrict the position of the urethral-insertion portion 31 in a urethra is not limited to the balloon 61. For example, the urethral-insertion portion 31 that partially curves, an object that holds part of the urinary tract tissue surface, and so forth are also possible.

Furthermore, on the surface of the middle portion of the urethral-insertion portion 31 on the side of the vaginal-insertion portion 2, the reflective portion 36 that reflects ultrasound transmitted from the ultrasonic sensor 71 is provided.

Furthermore, the reflective portion 36 is not particularly limited as long as it can reflect ultrasound. For example, as its constituent material, an object obtained by using a metal material such as stainless steel, aluminum or aluminum alloy, or titanium or titanium alloy and forming plural minute concavities and convexities on a surface, and so forth can be used. In addition, if the urethral-insertion portion 31 is composed of a metal material, plural minute concavities and convexities may be formed on a surface of the urethral-insertion portion 31 as the reflective portion 36.

Moreover, on the surface of the middle portion of the vaginal-insertion portion 2 on the side of the urethral-insertion portion 31, the reflective portion 21 that reflects ultrasound transmitted from the ultrasonic sensor 72 is provided.

Furthermore, the reflective portion 21 is not particularly limited as long as it can reflect ultrasound. For example, as its constituent material, an object obtained by using a metal material such as stainless steel, aluminum or aluminum alloy, or titanium or titanium alloy and forming plural minute concavities and convexities on a surface, and so forth can be used. In addition, if the vaginal-insertion portion 2 is composed of a metal material, plural minute concavities and convexities may be formed on a surface of the vaginal-insertion portion 2 as the reflective portion 21.

In accordance with an exemplary embodiment, although the reflective portion 36 is provided on part of the surface of the urethral-insertion portion 31 in the present exemplary embodiment, the configuration is not limited thereto, and for example, the reflective portion 36 may be provided on the whole surface of the urethral-insertion portion 31. In this case, the shortest distance between the urethral-insertion portion 31 and the site where the ultrasonic sensor 71 is placed on the puncture needle assembly 9 is detected by the ultrasonic sensor 71.

Furthermore, although the reflective portion 21 is provided on part of the surface of the vaginal-insertion portion 2 in the present exemplary embodiment, the configuration is not limited thereto, and for example, the reflective portion 21 may be provided on the whole surface of the vaginal-insertion portion 2. In this case, the shortest distance between the vaginal-insertion portion 2 and the site where the ultrasonic sensor 72 to be described later is placed on the puncture needle assembly 9 is detected by the ultrasonic sensor 72.

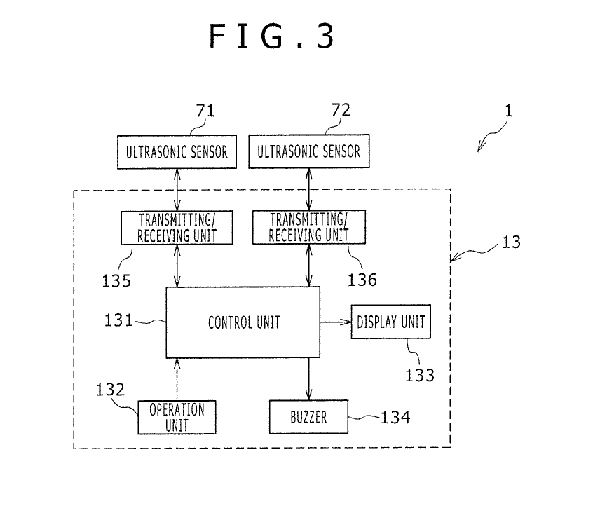

As illustrated in FIG. 3, the apparatus main body 13 has a control unit 131, an operation unit 132 to carry out respective operations, a display unit 133 as informing means, a buzzer 134 as informing means, and transmitting/receiving units 135 and 136 that perform signal transmission and reception to and from the ultrasonic sensors 71 and 72. The transmitting/receiving units 135 and 136 of this apparatus main body 13 are electrically connected to the ultrasonic sensors 71 and 72, respectively, via cables not illustrated.

The control unit 131 is formed of for example, a microcomputer or the like and accepts an input signal from the operation unit 132. Furthermore, the control unit 131 carries out control of the whole of the puncture apparatus 1, such as the display unit 133, the buzzer 134, and the transmitting/receiving units 135 and 136. In accordance with an exemplary embodiment detection means that detects information relating to the positional relationship between the distal portion of the puncture needle assembly 9 and the urethral-insertion portion 31, the vaginal-insertion portion 2, and the guide portion 102 is configured by the control unit 131, the transmitting/receiving units 135 and 136, and the ultrasonic sensors 71 and 72. Furthermore, determination means and comparison means are configured by the control unit 131.

From the operation unit 132, the respective inputs are made, such as measurement time ts, measurement interval Δt, maximum value Lvmax and minimum value Lvmin of the allowable range of separated distance Lv, and maximum value Lumax and minimum value Lumin of the allowable range of separated distance Lu.

The display unit 133 is not particularly limited, and, for example, liquid crystal display apparatus, CRT, can be used.

The transmitting/receiving units 135 and 136 transmit, to the ultrasonic sensors 71 and 72, an electrical signal for transmitting ultrasound from the ultrasonic sensors 71 and 72, respectively. Furthermore, when the ultrasonic sensors 71 and 72 receive ultrasound and an electrical signal is transmitted from the ultrasonic sensors 71 and 72, the transmitting/receiving units 135 and 136 receive the signal.

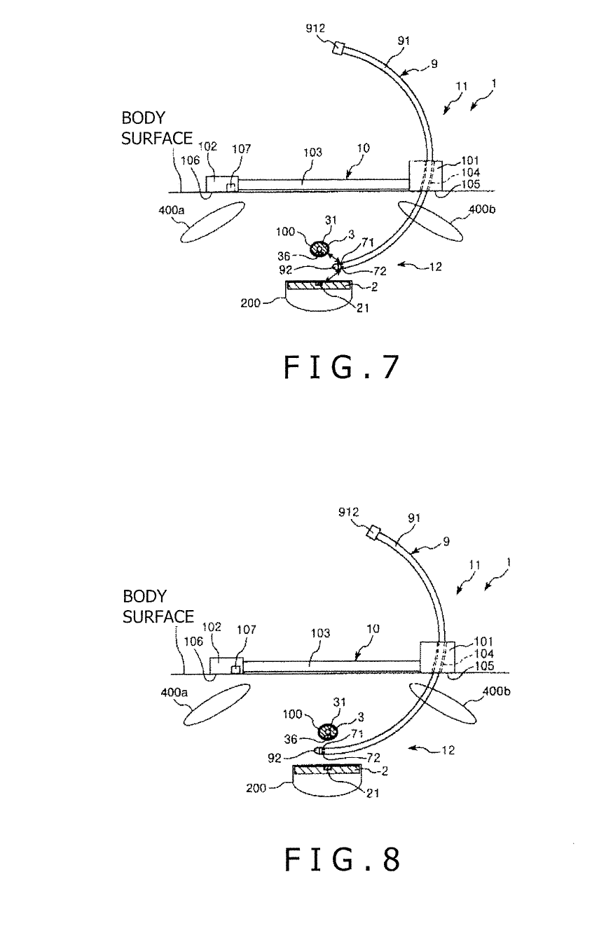

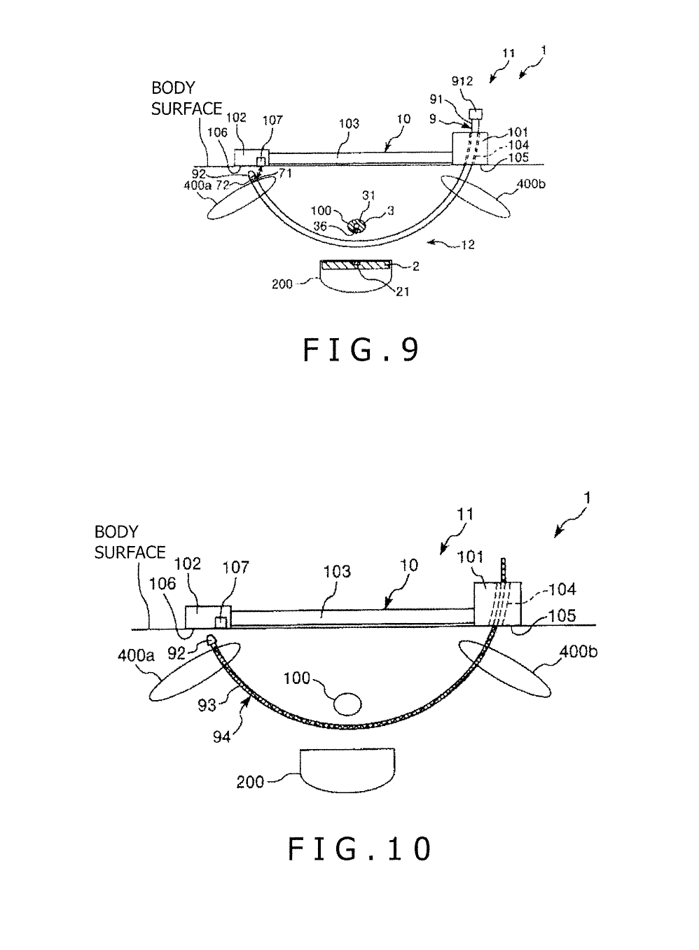

In accordance with an exemplary embodiment, the puncture hole formed in living body tissues by puncturing the living body tissues by the puncture needle assembly 9 of the puncture apparatus 1 is a non-through-hole that penetrates the body surface on the side of the support member 10 of the first apparatus 11 and does not penetrate the body surface on the side of the guide portion 102 (see FIGS. 9 and 10). In forming this puncture hole in the living body tissues, the puncture needle assembly 9 rotationally moves with the center O of the circular arc of the outer tube 91 being the center of the rotary movement, and the distal portion of the puncture needle assembly 9, for example, the needle tip 921, passes between the urethral-insertion portion 31 and the vaginal-insertion portion 2 (see FIGS. 7 and 8).

Furthermore, in this puncture apparatus 1, when living body tissues are punctured by the puncture needle assembly 9, the control unit 131 carries out transmission and reception of ultrasound by the ultrasonic sensors 71 and 72 to thereby detect information relating to each of the positional relationship between the distal portion of the puncture needle assembly 9 and the urethral-insertion portion 31, the positional relationship between the distal portion of the puncture needle assembly 9 and the vaginal-insertion portion 2, and the positional relationship between the distal portion of the puncture needle assembly 9 and the guide portion 102 of the support member 10. In accordance with an exemplary embodiment, because the urethral-insertion portion 31 is inserted into the urethra of a patient, the positional relationship between the distal portion of the puncture needle assembly 9 and the urethral-insertion portion 31 is equivalent to the positional relationship between the distal portion of the puncture needle assembly 9 and the urethral wall. Furthermore, because the vaginal-insertion portion 2 is inserted into the vagina of the patient, the positional relationship between the distal portion of the puncture needle assembly 9 and the vaginal-insertion portion 2 is equivalent to the positional relationship between the distal portion of the puncture needle assembly 9 and the vaginal wall. In addition, because the surface 106 of the guide portion 102 is placed on the body surface of the patient, the positional relationship between the distal portion of the puncture needle assembly 9 and the guide portion 102 is equivalent to the positional relationship between the distal portion of the puncture needle assembly 9 and the skin.

In accordance with an exemplary embodiment, the control unit 131 transmits ultrasound by the ultrasonic sensor 71. This ultrasound reflects at the reflective portion 36 provided on the urethral-insertion portion 31 and is received by the ultrasonic sensor 71. Then, the control unit 131 measures the time from the transmission of the ultrasound by the ultrasonic sensor 71 to the reception and obtains the separated distance Lu between the distal portion of the puncture needle assembly 9 and the urethral-insertion portion 31. In accordance with an exemplary embodiment, the allowable range of the separated distance Lu, specifically the maximum value Lumax and the minimum value Lumin of the allowable range of the separated distance Lu, is set in advance.

The maximum value Lumax is not particularly limited and is appropriately set according to conditions. For example, it can be preferable that the maximum value Lumax is about 15 mm to 50 mm, and, for example, it can be more preferable that the maximum value Lumax is about 20 mm to 40 mm.

In addition, the minimum value Lumin is not particularly limited and is appropriately set according to conditions. For example, it can be preferable that the minimum value Lumin is about 1 mm to 10 mm, and, for example, it can be more preferable that the minimum value Lumin is about 3 mm to 8 mm.

Furthermore, the control unit 131 transmits ultrasound by the ultrasonic sensor 72. This ultrasound reflects at the reflective portion 21 provided on the vaginal-insertion portion 2 and is received by the ultrasonic sensor 72. Then, the control unit 131 measures the time from the transmission of the ultrasound by the ultrasonic sensor 72 to the reception and obtains the separated distance Lv between the distal portion of the puncture needle assembly 9 and the vaginal-insertion portion 2. In accordance with an exemplary embodiment, the allowable range of the separated distance Lv, specifically the maximum value Lvmax and the minimum value Lvmin of the allowable range of the separated distance Lv, can be set in advance.

The maximum value Lvmax is not particularly limited and is appropriately set according to conditions. For example, it can be preferable that the maximum value Lvmax is about 15 mm to 50 mm, and, for example, it can be more preferable that the maximum value Lvmax is about 20 mm to 40 mm.

In addition, the minimum value Lvmin is not particularly limited and is appropriately set according to conditions. For example, it can be preferable that the minimum value Lvmin is about 1 mm to 10 mm, and, for example, it is more preferable that the minimum valve Lvmin is about 3 mm to 8 mm.

If these separated distances Lu and Lv are each in the allowable range, living body tissues can be properly punctured by the puncture needle assembly 9. In this case, the distal portion of the puncture needle assembly 9, for example, the needle tip 921, passes between the urethral-insertion portion 31 and the vaginal-insertion portion 2 and can be prevented from puncturing the urethra and the vagina.

In accordance with an exemplary embodiment, the configuration can be made that, if the separated distances Lu and Lv are each in the allowable range, this status can be transmitted to the operator through no display is made on the display unit 133 and the buzzer 134 does not sound. However, the configuration is not limited thereto. For example, if the separated distances Lu and Lv are each in the allowable range, this status can be transmitted by making a predetermined displaying on the display unit 133.

In accordance with an exemplary embodiment, if the separated distance Lu is out of the allowable range, specifically if the separated distance Lu is smaller than the minimum value Lumin of the allowable range, the distance between the distal portion of the puncture needle assembly 9 and the urethral-insertion portion 31, for example, the distance between the distal portion of the puncture needle assembly 9 and the urethra, is too small, and there is a possibility that the urethra is punctured by the puncture needle assembly 9. Thus, a predetermined alarm can be made by each of the display unit 133 and the buzzer 134. Specifically, predetermined alarm information can be displayed on the display unit 133 and the buzzer 134 can be sounded, which can allow the operator to understand the situation and prevent the puncture needle assembly 9 from puncturing the urethra.

Furthermore, if the separated distance Lu is larger than the maximum value Lumax of the allowable range, the distance between the distal portion of the puncture needle assembly 9 and the urethral-insertion portion 31, for example, the distance between the distal portion of the puncture needle assembly 9 and the urethra, is too large, and there is a possibility that the state is not the normal state. Thus, a predetermined alarm can be made by each of the display unit 133 and the buzzer 134. Specifically, predetermined alarm information can be displayed on the display unit 133 and the buzzer 134 can be sounded, which can allow the operator to understand the situation and respond to it.

Moreover, if the separated distance Lv is out of the allowable range, specifically if the separated distance Lv is smaller than the minimum value Lvmin of the allowable range, the distance between the distal portion of the puncture needle assembly 9 and the vaginal-insertion portion 2, for example, the distance between the distal portion of the puncture needle assembly 9 and the vagina, is too small, and there is a possibility that the vaginal wall is punctured by the puncture needle assembly 9. Thus, a predetermined alarm can be made by each of the display unit 133 and the buzzer 134. Specifically, predetermined alarm information can be displayed on the display unit 133 and the buzzer 134 can be sounded, which can allow the operator to understand the situation and prevent the puncture needle assembly 9 from puncturing the vaginal wall.

Furthermore, if the separated distance Lv is larger than the maximum value Lvmax of the allowable range, the distance between the distal portion of the puncture needle assembly 9 and the vaginal-insertion portion 2, for example, the distance between the distal portion of the puncture needle assembly 9 and the vagina, is too large, and there is a possibility that the state is not the normal state. Thus, a predetermined alarm can be made by each of the display unit 133 and the buzzer 134. Specifically, predetermined alarm information can be displayed on the display unit 133 and the buzzer 134 can be sounded, which can allow the operator to understand the situation and respond to it.

Furthermore, after the distal portion of the puncture needle assembly 9 has passed between the urethral-insertion portion 31 and the vaginal-insertion portion 2, the control unit 131 continuously transmits ultrasound by the ultrasonic sensor 71. This ultrasound reflects at the reflective portion 107 provided on the guide portion 102 of the support member 10 and is received by the ultrasonic sensor 71. Then, the control unit 131 measures the time from the transmission of the ultrasound by the ultrasonic sensor 71 to the reception and obtains the separated distance Lw between the distal portion of the puncture needle assembly 9 and the guide portion 102. The control unit 131 determines the timing when the values of the detected separated distances Lu and Lv become the minimum values after gradual decrease as the timing when the distal portion of the puncture needle assembly 9 is passing between the urethral-insertion portion 31 and the vaginal-insertion portion 2. In accordance with an exemplary embodiment, a target value Lwp of the separated distance Lw is so set in advance that the needle tip 921 of the puncture needle assembly 9 comes close to, and does not reach, the surface 106 of the guide portion 102, for example, the body surface, when the detection value of the separated distance Lw becomes the target value Lwp.

The target value Lwp is not particularly limited and is appropriately set according to conditions. For example, it can be preferable that the target value Lwp is about 5 mm to 60 mm, and, for example, it can be more preferable that the target value Lwp is about 10 mm to 40 mm.

When the separated distance Lw becomes the target value Lwp, the control unit 131 carries out predetermined informing by each of the display unit 133 and the buzzer 134. Specifically, predetermined information can be displayed on the display unit 133 and the buzzer 134 can be sounded, which can allow the operator to understand that the distal portion of the puncture needle assembly 9 is located near the guide portion 102. For example, in this case, the operator can stop the rotary movement of the puncture needle assembly 9, which can help prevent the puncture needle assembly 9 from penetrating the body surface on the side of the guide portion 102 and forming the non-through-hole in living body tissues.

In accordance with an exemplary embodiment, the configuration may be so made that the separated distances Lu, Lv, and Lw are always displayed on the display unit 133. This is the same also in the respective embodiments to be described later.

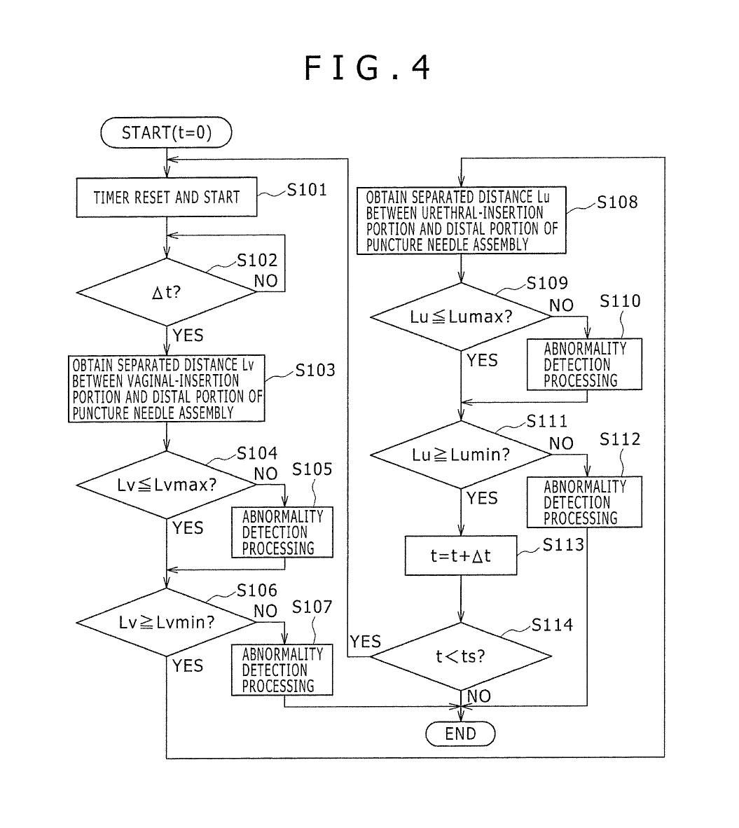

Next, regarding the control operation of the control unit 131 of the puncture apparatus 1, representatively a case of detecting the separated distance Lu between the distal portion of the puncture needle assembly 9 and the urethral-insertion portion 31 and the separated distance Lv between the distal portion of the puncture needle assembly 9 and the vaginal-insertion portion 2 will be described based on the flowchart illustrated in FIG. 4.

First, the measurement time ts during which measurement is performed, the measurement interval Δt, the maximum value Lvmax and the minimum value Lvmin of the allowable range of the separated distance Lv, and the maximum value Lumax and the minimum value Lumin of the allowable range of the separated distance Lu are set in advance. In accordance with an exemplary embodiment, these measurement time ts, measurement interval Δt, maximum value Lvmax, minimum value Lvmin, maximum value Lumax, and minimum value Lumin can be each arbitrarily set by the operation unit 132. Then, when a start button, not illustrated, of the operation unit 132 is pressed, the control unit 131 carries out control described below.

In accordance with an exemplary embodiment, the measurement time ts is not particularly limited and is appropriately set according to conditions. For example, it can be preferable that the measurement time ts is about 4 seconds to 60 seconds, and, for example, it is more preferable that the measurement time ts is about 10 seconds to 30 seconds.

Furthermore, the measurement interval Δt is not particularly limited and is appropriately set according to conditions. For example, it can be preferable that the measurement interval Δt is about 0.1 seconds to 2 seconds, and, for example, it can be more preferable that the measurement interval Δt is about 0.2 seconds to 1 second.

As illustrated in FIG. 4, time t indicating the elapsed time from the start of this control can be set to 0 and the control unit 131 first resets and starts a timer not illustrated to start measurement of the measurement interval Δt (step S101).

Subsequently, the control unit 131 determines whether the time t has reached the measurement interval Δt (step S102). If the time t has reached the measurement interval Δt, the control unit 131 obtains the separated distance Lv (step S103).

In the step S103, the control unit 131 carries out transmission and reception of ultrasound by the ultrasonic sensor 72 and obtains the time between the timing of the transmission of the ultrasound and the timing of the reception. Based on the time, the control unit 131 can calculate the separated distance Lv.

Subsequently, the control unit 131 can determine whether or not the obtained separated distance Lv is equal to or smaller than the maximum value Lvmax of the allowable range (step S104). If the separated distance Lv is larger than the maximum value Lvmax of the allowable range, the control unit 131 executes abnormality detection processing (step S105).

In this abnormality detection processing, the control unit 131 displays alarm information on the display unit 133 and sounds the buzzer 134 for example. As the alarm information, for example, “the needle exists at a position remote from the vagina,” “please check whether the vaginal-insertion portion is correctly set,” can be used.

In accordance with an exemplary embodiment, the operator can know the existence of some kind of abnormality due to the sounding of the buzzer 134 and can understand the contents of the abnormality, coping methods, and so forth according to the contents of the displaying on the display unit 133.

Furthermore, if the separated distance Lv is equal to or smaller than the maximum value Lvmax of the allowable range in the step S104, the control unit 131 can determine whether or not the obtained separated distance Lv is equal to or larger than the minimum value Lvmin of the allowable range (step S106). If the separated distance Lv is smaller than the minimum value Lvmin of the allowable range, the control unit 131 executes the abnormality detection processing (step S107) to end this program.

In this abnormality detection processing, the control unit 131 displays alarm information on the display unit 133 and sounds the buzzer 134 for example. As the alarm information, for example, “the needle exists at a position close to the vagina,” “please temporarily stop puncture operation of the needle and check the state of the vagina,” can be used.

In accordance with an exemplary embodiment, the operator can know the existence of some kind of abnormality due to the sounding of the buzzer 134 and can understand the contents of the abnormality, coping methods, and so forth according to the contents of the displaying on the display unit 133.

Furthermore, if the obtained separated distance Lv is equal to or larger than the minimum value Lvmin of the allowable range in the step S106, the control unit 131 can obtain the separated distance Lu (step S108).

In the step S108, the control unit 131 carries out transmission and reception of ultrasound by the ultrasonic sensor 71 and obtains the time between the timing of the transmission of the ultrasound and the timing of the reception. Based on the time, the control unit 131 calculates the separated distance Lu.

Subsequently, the control unit 131 determines whether or not the obtained separated distance Lu is equal to or smaller than the maximum value Lumax of the allowable range (step S109). If the separated distance Lu is larger than the maximum value Lumax of the allowable range, the control unit 131 executes the abnormality detection processing (step S110).

In this abnormality detection processing (step S110), the control unit 131 displays alarm information on the display unit 133 and sounds the buzzer 134 for example. As the alarm information, for example, “the needle exists at a position remote from the urethra,” “please check whether the urethral-insertion portion is correctly set,” can be used.

In accordance with an exemplary embodiment, the operator can know the existence of some kind of abnormality due to the sounding of the buzzer 134 and can understand the contents of the abnormality, coping methods, and so forth according to the contents of the displaying on the display unit 133.

Furthermore, if the separated distance Lu is equal to or smaller than the maximum value Lumax of the allowable range in the step S109, the control unit 131 determines whether or not the obtained separated distance Lu is equal to or larger than the minimum value Lumin of the allowable range (step S111). If the separated distance Lu is smaller than the minimum value Lumin of the allowable range, the control unit 131 executes the abnormality detection processing (step S112) to end this program.

In this abnormality detection processing, the control unit 131 can display alarm information on the display unit 133 and sound the buzzer 134 for example. As the alarm information, for example, “the needle exists at a position close to the urethra,” “please temporarily stop puncture operation of the needle and check the state of the urethra,” can be used.

In accordance with an exemplary embodiment, the operator can know the existence of some kind of abnormality due to the sounding of the buzzer 134 and can understand the contents of the abnormality, coping methods, and so forth according to the contents of the displaying on the display unit 133.

Furthermore, if the obtained separated distance Lu is equal to or larger than the minimum value Lumin of the allowable range in the step S111, the control unit 131 proceeds to the step S113. In accordance with an exemplary embodiment, the separated distance Lu falls within the allowable range in this case.

Subsequently, the control unit 131 adds the measurement interval Δt to the time t (step S113).

Next, the control unit 131 determines whether or not, the time t is smaller than the measurement time ts (step S114). If the time t is smaller than the measurement time ts, the control unit 131 returns to the step S101 and carries out the step S101 and the subsequent steps again. That is, the control unit 131 carries out the respective steps of the step S103 and the subsequent steps at a time interval of Δt.

Furthermore, if the time t is equal to or larger than the measurement time ts in the step S114, the control unit 131 ends this program.

In accordance with an exemplary embodiment, the display color of the display unit 133 may be changed from that in normal time and displaying on the display unit 133 may blink, for example, when there is a need to call operator's attention, for example, in each of the steps of the abnormality detection processing.

Furthermore, in the respective steps of the abnormality detection processing, the pitches of the sound of the buzzer 134 and the patterns of the sound may be identical to each other or may be different from each other.

Next, one example of the use method of the puncture apparatus 1 will be described with reference to FIGS. 5 to 12. Here, the description will be made about procedure until the inside structural body 94 having the implant 93 for treatment of urinary incontinence of a woman is implanted in a living body by using the puncture apparatus 1. In this procedure, the puncture hole formed by the puncture apparatus 1 is a non-through-hole formed from an obturator foramen 400b on the right side toward an obturator foramen 400a on the left side.

As illustrated in FIG. 5, the puncture apparatus 1 is set to the use state. Specifically, the puncture apparatus 1 is set to a state in which the surface 105 of the support portion 101 in the support member 10 of the first apparatus 11 is placed on the body surface and the surface 106 of the guide portion 102 is put on the body surface. In addition, the puncture apparatus 1 is set to a state in which the urethral-insertion portion 31 of the second apparatus 12 is inserted into a urethra 100 and the vaginal-insertion portion 2 is inserted into a vagina 200. In accordance with an exemplary embodiment, in the use state, the support portion 101 is put on the portion of the body surface corresponding to (above) the obturator foramen 400b on the right side and the guide portion 102 is put on the portion of the body surface corresponding to (above) the obturator foramen 400a on the left side.

Furthermore, the balloon 61 is inserted into the bladder in the deflated state and a balloon inflation device such as a syringe not illustrated is connected to the port 34. Then, a working fluid supplied from the balloon inflation device is sent to the inside of the balloon 61 via the lumen 33 to inflate the balloon 61. Due to this, the inflated balloon 61 gets caught on the bladder neck and thereby the position of the urethral-insertion portion 31 relative to the bladder and the urethra can be fixed.

In accordance with an exemplary embodiment, the position of the urethral-insertion portion 31 relative to the interlock portion 4 is changed to adjust the separated distance between the vaginal-insertion portion 2 and the urethral-insertion portion 31 according to need. Specifically, according to need, the male screw 51 is rotated in the loosening direction and the urethral-insertion member 3 is moved toward the left side or right side in FIG. 2(a) relative to the interlock portion 4. Then, the male screw 51 is rotated in the fastening direction to make a state in which the movement of the urethral-insertion member 3 relative to the interlock portion 4 is inhibited.

Next, as illustrated in FIG. 6, the puncture needle assembly 9 in the assembled state is inserted into the through-hole 104 of the support portion 101 from the side of the needle tip 921.

Then, as illustrated in FIGS. 7 to 9, the puncture needle assembly 9 is thrust directly and rotary movement operation of the puncture needle assembly 9 can be carried out from the side of the support portion 101 toward the side of the guide portion 102. At this time, the positional relationship among the puncture needle assembly 9, the urethral-insertion portion 31, and the vaginal-insertion portion 2 is restricted by the support portion 101 so that the needle tip 921 may pass through the remoter side from the center O of the circular arc of the outer tube 91 (lower side in FIG. 7) than the urethral-insertion portion 31, for example, the needle tip 921 may pass between the urethral-insertion portion 31 and the vaginal-insertion portion 2. Due to this, the puncture hole formed by the puncture needle assembly 9 is a hole passing through a very thin membrane of living body tissues between the urethra 100 and the vagina 200.

In accordance with an exemplary embodiment, as described above, in the puncture apparatus 1, transmission and reception of ultrasound can be carried out by the ultrasonic sensors 71 and 72 and the aforementioned respective steps of processing are carried out based on control by the control unit 131.

As illustrated in FIG. 7, the distal portion of the puncture needle assembly 9 thereby comes close to the urethral-insertion portion 31 and the vaginal-insertion portion 2 and the separated distance Lu between the distal portion of the puncture needle assembly 9 and the urethral-insertion portion 31 and the separated distance Lv between the distal portion of the puncture needle assembly 9 and the vaginal-insertion portion 2 are detected. In each of a case in which the separated distance Lu is too small and a case in which the separated distance Lv is too small, predetermined alarm information can be displayed on the display unit 133 and the buzzer 134 can be sounded. In this case, for example, the operator can temporarily remove the puncture needle assembly 9 and change the position of the support member 10 to change the trajectory of the puncture needle assembly 9, which can help prevent the puncture needle assembly 9 from puncturing the urethra and puncturing the vaginal wall.

Furthermore, as illustrated in FIG. 9, when the separated distance Lw between the distal portion of the puncture needle assembly 9 and the guide portion 102 is detected and the separated distance Lw becomes the target value Lwp, predetermined information is displayed on the display unit 133 and the buzzer 134 is sounded. Thus, the operator ends the rotary movement operation of the puncture needle assembly 9, which can help prevent the puncture needle assembly 9 from penetrating the body surface on the side of the guide portion 102 and forming a non-through-hole as the puncture hole for implanting the implant 93 in living body tissues.

Next, as illustrated in FIG. 10, only the outer tube 91 is pulled from the puncture needle assembly 9 in the opposite direction of the aforementioned direction and the outer tube 91 is withdrawn. The puncture needle assembly 9 thereby becomes the decomposed state and only the inside structural body 94 is indwelled in living body tissues. Furthermore, by engagement of the step portion 922 of the needle body 92 with living body tissues, this inside structural body 94 is prevented from involuntarily dropping off from living body issues.

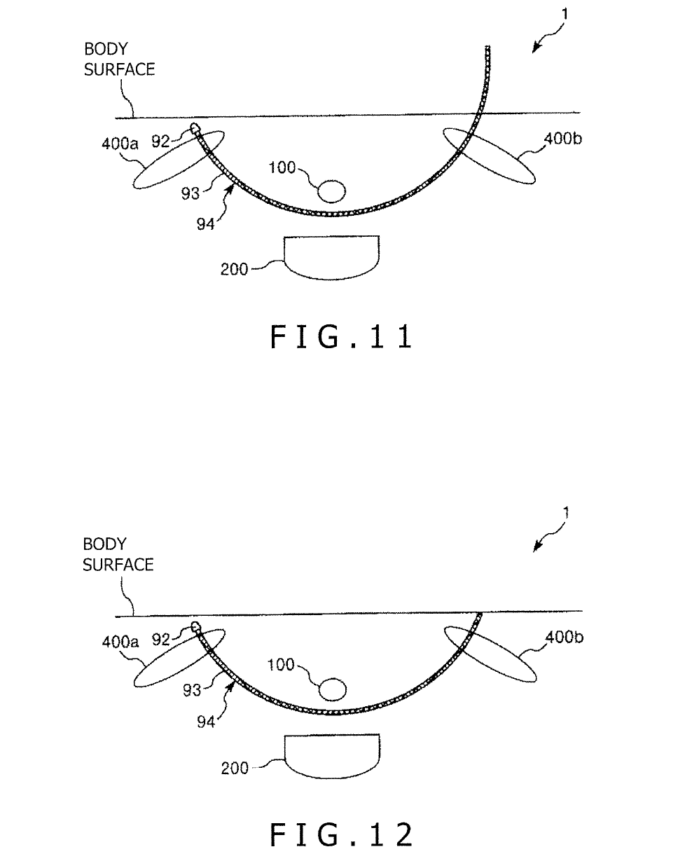

Next, as illustrated in FIG. 11, the support member 10 is removed from the body surface of the patient and the second apparatus 12 is detached from the patient.

Next, as illustrated in FIG. 12, the length of the implant 93 of the inside structural body 94 is adjusted to such a degree as to be able to support the urethra 100 from the lower side in FIG. 12, and the portion of the implant 93 on the opposite side to the needle body 92 is fixed to the body surface. Thereafter, the unnecessary portion of the implant 93 can be cut off and predetermined suture can be performed to end the procedure.

As described above, according to this puncture apparatus 1, in implanting the implant 93 in the living body, it is possible to deal with the implanting only through a less-invasive procedure such as puncture with the puncture needle assembly 9 and a highly-invasive incision or the like does not need to be performed. Therefore, the burden on the patient can be relatively light and the safety of the patient can be relatively high.

Furthermore, if the separated distance Lu between the distal portion of the puncture needle assembly 9 and the urethral-insertion portion 31 is too small or if the separated distance Lv between the distal portion of the puncture needle assembly 9 and the vaginal-insertion portion 2 is too small, predetermined alarm information can be displayed on the display unit 133 of the apparatus main body 13 and the buzzer 134 can be sounded. Thus, the situation can be understood by the operator, which can help prevent the puncture needle assembly 9 from puncturing the urethra and puncturing the vaginal wall, providing safety. In addition, the target region of living body tissues can be punctured by the puncture needle assembly 9 and the implant 93 can be implanted in the target region.

Moreover, when the separated distance Lw between the distal portion of the puncture needle assembly 9 and the guide portion 102 becomes the target value Lwp, predetermined information can be displayed on the display unit 133 and the buzzer 134 can be sounded. Thus, the puncture needle assembly 9 can be prevented from penetrating the body surface on the side of the guide portion 102 and a non-through-hole can be formed as the puncture hole for implanting the implant 93 in living body tissues.

In addition, the operator does not need to perform an incision or the like, which can prevent damage to a fingertip by a surgical knife or the like, providing additional safety for the operator.

In accordance with an exemplary embodiment, although the puncture hole formed in the patient by the puncture needle assembly 9 is a hole that does not penetrate the living body surface on one side in the present embodiment, the configuration is not limited thereto and the puncture hole may be a through-hole that penetrates the living body surface on both sides.

Furthermore, in the present embodiment, the whole of the puncture needle assembly (puncture needle) is curved into a circular arc shape. However, the configuration is not limited thereto and the puncture needle assembly may be a component only partially having a region curved into a circular arc shape for example. For example, the puncture needle assembly can at least partially have a region curved into a circular arc shape.

Moreover, the puncture needle assembly may be any component as long as it is a component at least partially having a curved region. For example, the puncture needle assembly may be a component totally curved into an elliptical arc shape or a component only partially having a region curved into an elliptical arc shape. For example, the puncture needle assembly may be a component at least partially having a region curved into an elliptical arc shape.

In addition, the puncture needle assembly may be a component that does not have a curved region, for example, a component forming a straight line shape.

Furthermore, the informing means is not limited to the display unit and the buzzer. In accordance with an exemplary embodiment, for example, a unit that informs information by voice and so forth can be used.

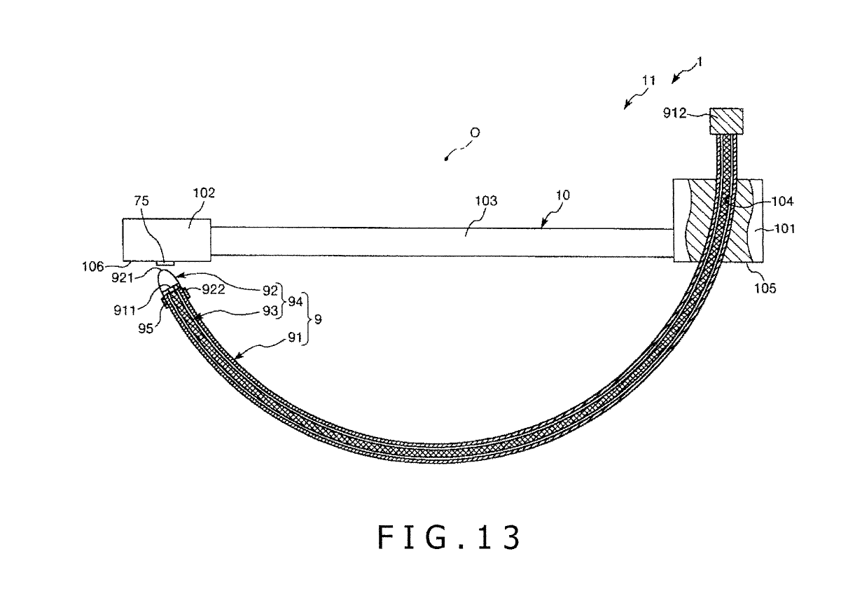

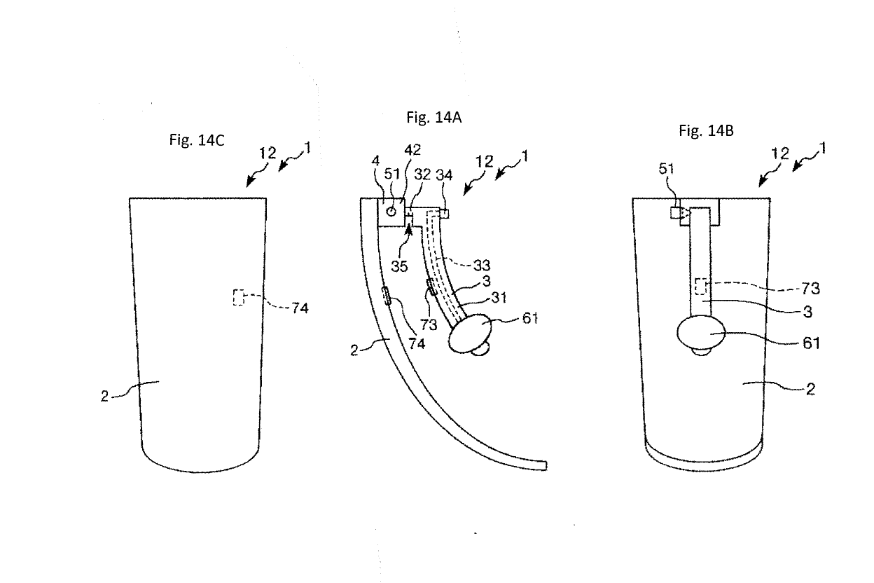

In accordance with an exemplary embodiment, in the following, description will be so made that the left side is defined as the “distal end” and the right side is defined as the “proximal end” along the longitudinal direction of a puncture needle assembly in FIG. 13 and the lower side is defined as the “distal end” and the upper side is defined as the “proximal end” along the longitudinal direction of a vaginal-insertion portion and a urethral-insertion portion in FIG. 14.

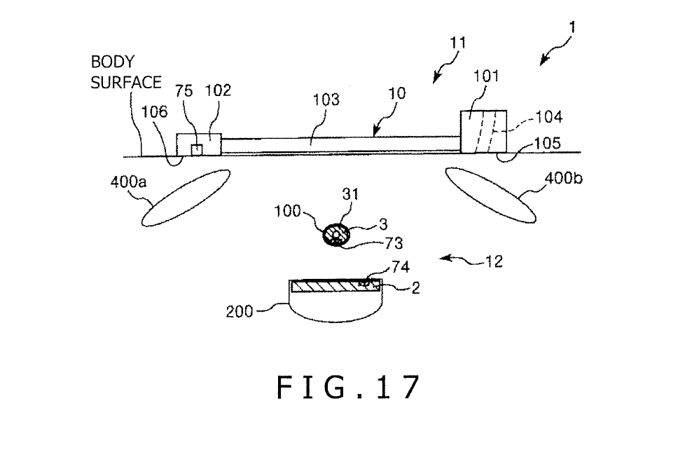

Furthermore, in each of FIGS. 17 to 24, for high visibility, each ultrasonic sensor and each reflective portion are represented with a large size schematically and slant lines in the living body are omitted.

In the following, the second exemplary embodiment will be described mainly about differences from the aforementioned first exemplary embodiment and description of similar matters will be omitted.

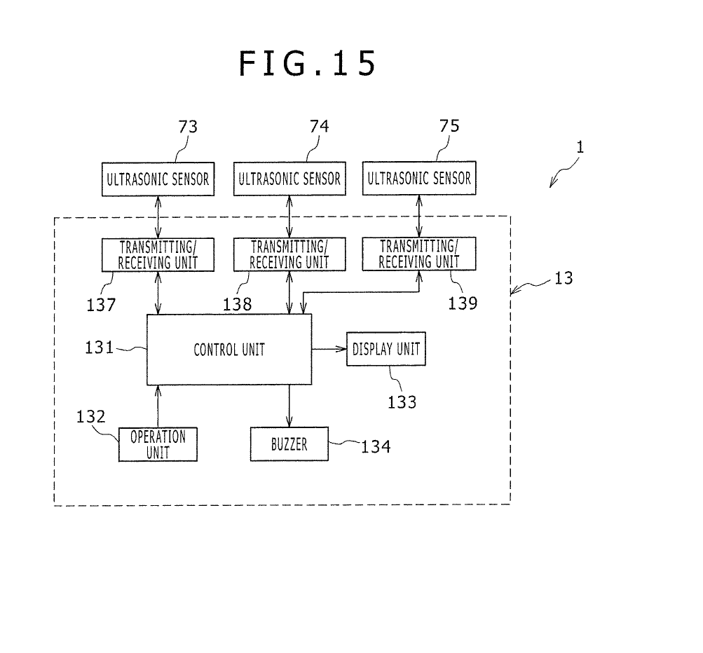

As illustrated in FIGS. 13 to 15, in puncture apparatus 1 of the second exemplary embodiment, a reflective portion 95 that reflects each of ultrasounds transmitted from ultrasonic sensors 73, 74, and 75 to be described later is provided at the distal portion of the puncture needle assembly 9, for example, the distal portion of the outer tube 91. This reflective portion 95 is provided on the outer circumferential surface of the outer tube 91 across the whole round. However, the circumferential length thereof may be smaller than the whole round.

In accordance with an exemplary embodiment, the configuration of the reflective portion 95 is the same as the reflective portions 21 and 36 and therefore description of the reflective portion 95 is omitted.

Furthermore, the ultrasonic sensor 75 is placed on the surface 106 of the guide portion 102 of the support member 10. This ultrasonic sensor 75 transmits ultrasound toward the lower side in FIG. 13. The position of this ultrasonic sensor 75 is not particularly limited. In the present exemplary embodiment, the ultrasonic sensor 75 is disposed at the center portion of the guide portion 102.

In addition, the ultrasonic sensor 73 can be placed on the surface of the middle portion of the urethral-insertion portion 31 on the side of the vaginal-insertion portion 2. This ultrasonic sensor 73 transmits ultrasound toward the side of the vaginal-insertion portion 2, for example, the side of the ultrasonic sensor 74 to be described later.

Moreover, the ultrasonic sensor 74 is provided on the surface of the middle portion of the vaginal-insertion portion 2 on the side of the urethral-insertion portion 31. This ultrasonic sensor 74 transmits ultrasound toward the side of the urethral-insertion portion 31, for example, the side of the ultrasonic sensor 73.

Furthermore, the apparatus main body 13 has transmitting/receiving units 137, 138, and 139 that can perform signal transmission and reception to and from the ultrasonic sensors 73, 74, and 75, respectively.

In accordance with an exemplary embodiment, detection means that detects information relating to the positional relationship between the distal portion of the puncture needle assembly 9 and the urethral-insertion portion 31, the vaginal-insertion portion 2, and the guide portion 102 and information relating to the positional relationship between the urethral-insertion portion 31 and the vaginal-insertion portion 2 is configured by the control unit 131, the transmitting/receiving units 137 to 139, and the ultrasonic sensors 73 to 75.

In this puncture apparatus 1, the control unit 131 transmits ultrasound by the ultrasonic sensor 73 and receives the ultrasound by the ultrasonic sensor 74. Then, the control unit 131 measures the time from the transmission of the ultrasound by the ultrasonic sensor 73 to the reception of the ultrasound by the ultrasonic sensor 74 and obtains separated distance Lx between the urethral-insertion portion 31 and the vaginal-insertion portion 2.

In accordance with an exemplary embodiment, the control unit 131 may transmit ultrasound by the ultrasonic sensor 74 and receive the ultrasound by the ultrasonic sensor 73. The allowable range of the separated distance Lx, for example, maximum value Lxmax and minimum value Lxmin of the allowable range of the separated distance Lx, can be set in advance.

The maximum value Lxmax is not particularly limited and is appropriately set according to conditions. For example, it can be preferable that the maximum value Lxmax is about 15 mm to 50 mm, and, for example, it can be more preferable that the maximum value Lxmax is about 20 mm to 30 mm.

Furthermore, the minimum value Lxmin is not particularly limited and is appropriately set according to conditions. For example, it can be preferable that the minimum value Lxmin is about 5 mm to 14 mm, and, for example, it can be more preferable that the minimum value Lxmin is about 6 mm to 10 mm.

If this separated distance Lx is in the allowable range, living body tissues can be properly punctured by the puncture needle assembly 9. In this case, the distal portion of the puncture needle assembly 9, for example, the needle tip 921, passes between the urethral-insertion portion 31 and the vaginal-insertion portion 2 relatively easily and can be prevented from puncturing the urethra and the vagina. In accordance with an exemplary embodiment the configuration is so made that, if the separated distance Lx is in the allowable range, this can be informed through that no displaying is made on the display unit 133 and the buzzer 134 makes no sound in the present exemplary embodiment. However, the configuration is not limited thereto. For example, this may be informed by making predetermined displaying on the display unit 133.

On the other hand, if the separated distance Lx is out of the allowable range, for example, if the separated distance Lx is smaller than the minimum value Lxmin of the allowable range, the distance between the urethral-insertion portion 31 and the vaginal-insertion portion 2, for example, the distance between the urethra and the vagina, is too small, and there is a possibility that the urethra or the vagina is punctured by the puncture needle assembly 9. Thus, a predetermined alarm can be made by each of the display unit 133 and the buzzer 134. Specifically, for example, predetermined alarm information can be displayed on the display unit 133 and the buzzer 134 can be sounded, which can allow the operator to understand the situation and prevent the puncture needle assembly 9 from puncturing the urethra or the vagina.

Furthermore, if the separated distance Lx is larger than the maximum value Lxmax of the allowable range, the distance between the urethral-insertion portion 31 and the vaginal-insertion portion 2, for example, the distance between the urethra and the vagina, is large, and there is a possibility that the state is not the normal state. Thus, a predetermined alarm can be made by each of the display unit 133 and the buzzer 134. Specifically, for example, predetermined alarm information can be displayed on the display unit 133 and the buzzer 134 can be sounded, which can allow the operator to understand the situation and respond to it.

Moreover, in this puncture apparatus 1, ultrasound is transmitted by the ultrasonic sensor 73 and the ultrasound reflected by the reflective portion 95 is received by the ultrasonic sensor 73 to obtain separated distance Lu between the distal portion of the puncture needle assembly 9 and the urethral-insertion portion 31. Furthermore, ultrasound is transmitted by the ultrasonic sensor 74 and the ultrasound reflected by the reflective portion 95 is received by the ultrasonic sensor 74 to obtain separated distance Lv between the distal portion of the puncture needle assembly 9 and the vaginal-insertion portion 2. In addition, ultrasound is transmitted by the ultrasonic sensor 75 and the ultrasound reflected by the reflective portion 95 is received by the ultrasonic sensor 75 to obtain separated distance Lw between the distal portion of the puncture needle assembly 9 and the guide portion 102.

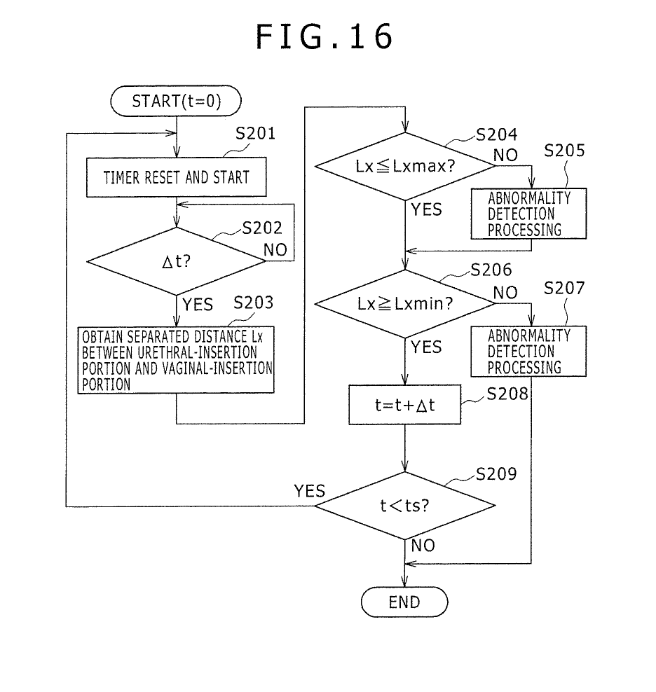

Next, the control operation of the control unit 131 of the puncture apparatus 1 in the case of detecting the separated distance Lx between the urethral-insertion portion 31 and the vaginal-insertion portion 2 will be described based on the flowchart illustrated in FIG. 16.

First, measurement time ts during which measurement is performed, measurement interval Δt, and maximum value Lxmax and minimum value Lxmin of the allowable range of the separated distance Lx can be set in advance. In accordance with an exemplary embodiment, these measurement time ts, measurement interval Δt, maximum value Lxmax, and minimum value Lxmin can each be arbitrarily set by the operation unit 132. Then, when a start button, not illustrated, of the operation unit 132 is pressed, the control unit 131 carries out control described below.

As illustrated in FIG. 16, time t indicating the elapsed time from the start of this control is set to 0 and the control unit 131 first resets and starts a timer not illustrated to start measurement of the measurement interval Δt (step S201).

Subsequently, the control unit 131 determines whether or not the time t has reached the measurement interval Δt (step S202). If the time t has reached the measurement interval Δt, the control unit 131 obtains the separated distance Lx (step S203).

In the step S203, the control unit 131 carries out transmission and reception of ultrasound by the ultrasonic sensors 73 and 74 and obtains the time between the timing of the transmission of the ultrasound and the timing of the reception. Based on the time, the control unit 131 calculates the separated distance Lx.

Subsequently, the control unit 131 determines whether or not the obtained separated distance Lx is equal to or smaller than the maximum value Lxmax of the allowable range (step S204). If the separated distance Lx is larger than the maximum value Lxmax of the allowable range, the control unit 131 executes abnormality detection processing (step S205).

In this abnormality detection processing, the control unit 131 displays alarm information on the display unit 133 and sounds the buzzer 134 for example. As the alarm information, for example, “the distance between the urethra and the vagina is long,” “please check whether each insertion portion is correctly set,” can be used.

The operator can know the existence of some kind of abnormality due to the sounding of the buzzer 134 and can understand the contents of the abnormality, coping methods, and so forth according to the contents of the displaying on the display unit 133.

Furthermore, if the separated distance Lx is equal to or smaller than the maximum value Lxmax of the allowable range in the step S204, the control unit 131 determines whether or not the obtained separated distance Lx is equal to or larger than the minimum value Lxmin of the allowable range (step S206). If the separated distance Lx is smaller than the minimum value Lxmin of the allowable range, the control unit 131 executes the abnormality detection processing (step S207) to end this program.

In this abnormality detection processing, the control unit 131 displays alarm information on the display unit 133 and sounds the buzzer 134 for example. As the alarm information, for example, “the distance between the urethra and the vagina is short,” “please check the state of the urethra and the vagina before carrying out puncture operation of the needle,” can be used.

The operator can know the existence of some kind of abnormality due to the sounding of the buzzer 134 and can understand the contents of the abnormality, coping methods, and so forth according to the contents of the displaying on the display unit 133.

Furthermore, if the obtained separated distance Lx is equal to or larger than the minimum value Lxmin of the allowable range in the step S206, the control unit 131 adds the measurement interval Δt to the time t (step S208).

Next, the control unit 131 determines whether or not, the time t is smaller than the measurement time is (step S209). If the time t is smaller than the measurement time ts, the control unit 131 returns to the step S201 and carries out the step S201 and the subsequent steps again. That is, the control unit 131 carries out the respective steps of the step S203 and the subsequent steps at a time interval of Δt.

Furthermore, if the time t is equal to or larger than the measurement time ts in the step S209, the control unit 131 ends this program.

The control operation of the control unit 131 in the case of detecting the separated distance Lu between the distal portion of the puncture needle assembly 9 and the urethral-insertion portion 31 and the separated distance Lv between the distal portion of the puncture needle assembly 9 and the vaginal-insertion portion 2 is the same as the first embodiment. Therefore, description thereof is omitted.

In accordance with an exemplary embodiment, maximum value Lumax and minimum value Lumin of the allowable range of the separated distance Lu may be each automatically set based on the separated distance Lx between the urethral-insertion portion 31 and the vaginal-insertion portion 2.

In this case, for example, it can be preferable that the maximum value Lumax is set in a range of about 40% to 100% of the separated distance Lx, and, for example, it can be more preferable that the maximum value Lumax is set in a range of about 70 to 100%.

In addition, for example, it can be preferable that the minimum value Lumin is set in a range of about 5% to 25% of the separated distance Lx, and, for example, it can be more preferable that the minimum value Lumin is set in a range of about 10% to 20%.

Furthermore, maximum value Lvmax and minimum value Lvmin of the allowable range of the separated distance Lv may be each automatically set based on the separated distance Lx between the urethral-insertion portion 31 and the vaginal-insertion portion 2.

In this case, for example, it can be preferable that the maximum value Lvmax is set in a range of about 40% to 100% of the separated distance Lx, and, for example, it can be more preferable that the maximum value Lvmax is set in a range of about 70% to 100%.

In addition, for example, it can be preferable that the minimum value Lvmin is set in a range of about 5% to 25% of the separated distance Lx, and, for example, it can be more preferable that the minimum value Lvmin is set in a range of 10% to 20%.

As illustrated in FIG. 17, the puncture apparatus 1 is set to the use state. Specifically, the puncture apparatus 1 is set to a state in which the surface 105 of the support portion 101 in the support member 10 of the first apparatus 11 is placed on the body surface and the surface 106 of the guide portion 102 is put on the body surface. In addition, the puncture apparatus 1 can be set to a state in which the urethral-insertion portion 31 of the second apparatus 12 is inserted into a urethra 100 and the vaginal-insertion portion 2 is inserted into a vagina 200. Furthermore, a balloon inflation device such as a syringe not illustrated is connected to the port 34 and a working fluid supplied from the balloon inflation device is sent to the inside of the balloon 61 via the lumen 33 to inflate the balloon 61. In accordance with an exemplary embodiment, the position of the urethral-insertion portion 31 relative to the interlock portion 4 is changed to adjust the separated distance between the vaginal-insertion portion 2 and the urethral-insertion portion 31 according to need.

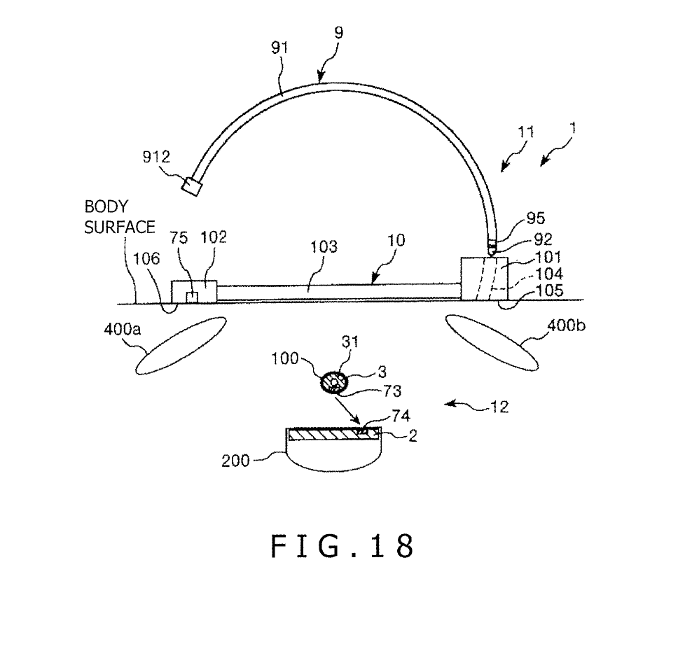

Next, as illustrated in FIG. 18, the puncture needle assembly 9 in the assembled state is inserted into the through-hole 104 of the support portion 101 from the side of the needle tip 921.

Then, as illustrated in FIGS. 19 to 21, the puncture needle assembly 9 is thrust directly and rotary movement operation of the puncture needle assembly 9 can be carried out from the side of the support portion 101 toward the side of the guide portion 102. At this time, the positional relationship among the puncture needle assembly 9, the urethral-insertion portion 31, and the vaginal-insertion portion 2 is restricted by the support portion 101 so that the needle tip 921 may pass through the remoter side from the center O of the circular arc of the outer tube 91 (lower side in FIG. 19) than the urethral-insertion portion 31, for example, the needle tip 921 may pass between the urethral-insertion portion 31 and the vaginal-insertion portion 2. Due to this, the puncture hole formed by the puncture needle assembly 9 is a hole passing through a very thin membrane of living body tissues between the urethra 100 and the vagina 200.

In accordance with an exemplary embodiment, as described above, in the puncture apparatus 1, transmission and reception of ultrasound are carried out by the ultrasonic sensors 73 and 74 and the aforementioned respective steps of processing are carried out based on control by the control unit 131.

First, the separated distance Lx between the urethral-insertion portion 31 and the vaginal-insertion portion 2 is thereby detected as illustrated in FIG. 18. If the separated distance Lx is too small, predetermined alarm information can be displayed on the display unit 133 and the buzzer 134 can be sounded. In this case, for example, the operator removes the second apparatus 12 from the patient and changes the position of the urethral-insertion portion 31 relative to the interlock portion 4 to adjust the separated distance Lx between the urethral-insertion portion 31 and the vaginal-insertion portion 2, which can help prevent the puncture needle assembly 9 from puncturing the urethra and puncturing the vaginal wall.

Furthermore, as illustrated in FIG. 19, the distal portion of the puncture needle assembly 9 comes close to the vaginal-insertion portion 2 and the separated distance Lv between the distal portion of the puncture needle assembly 9 and the vaginal-insertion portion 2 is detected. If the separated distance Lv is too small, predetermined alarm information can be displayed on the display unit 133 and the buzzer 134 is sounded. In this case, for example, the operator temporarily removes the puncture needle assembly 9 and changes the position of the support member 10 to change the trajectory of the puncture needle assembly 9, which can help prevent the puncture needle assembly 9 from puncturing the vaginal wall.

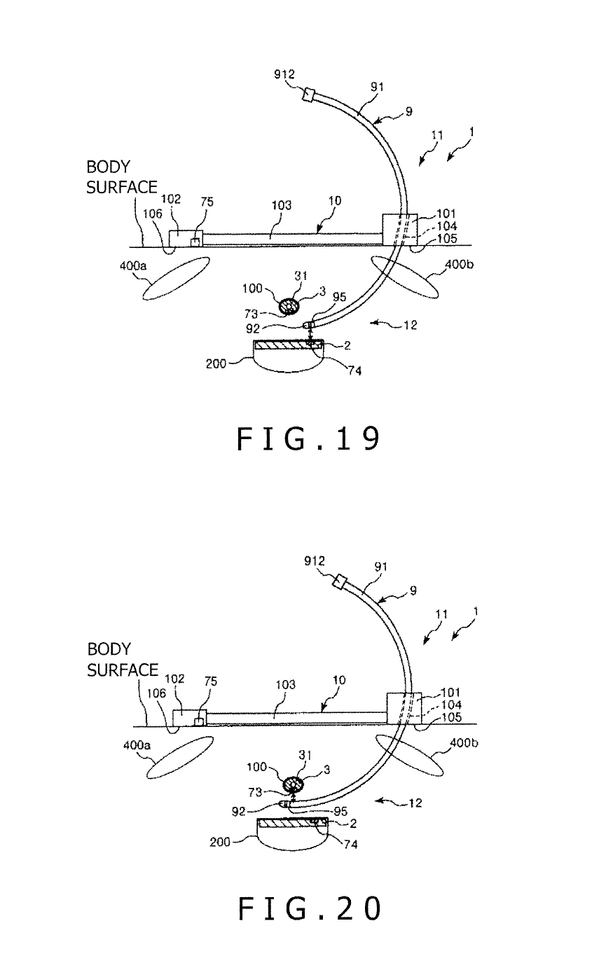

Moreover, as illustrated in FIG. 20, the distal portion of the puncture needle assembly 9 comes close to the urethral-insertion portion 31 and the separated distance Lu between the distal portion of the puncture needle assembly 9 and the urethral-insertion portion 31 is detected. If the separated distance Lu is too small, predetermined alarm information can be displayed on the display unit 133 and the buzzer 134 can be sounded. In this case, for example, the operator temporarily removes the puncture needle assembly 9 and changes the position of the support member 10 to change the trajectory of the puncture needle assembly 9, which can help prevent the puncture needle assembly 9 from puncturing the urethra.

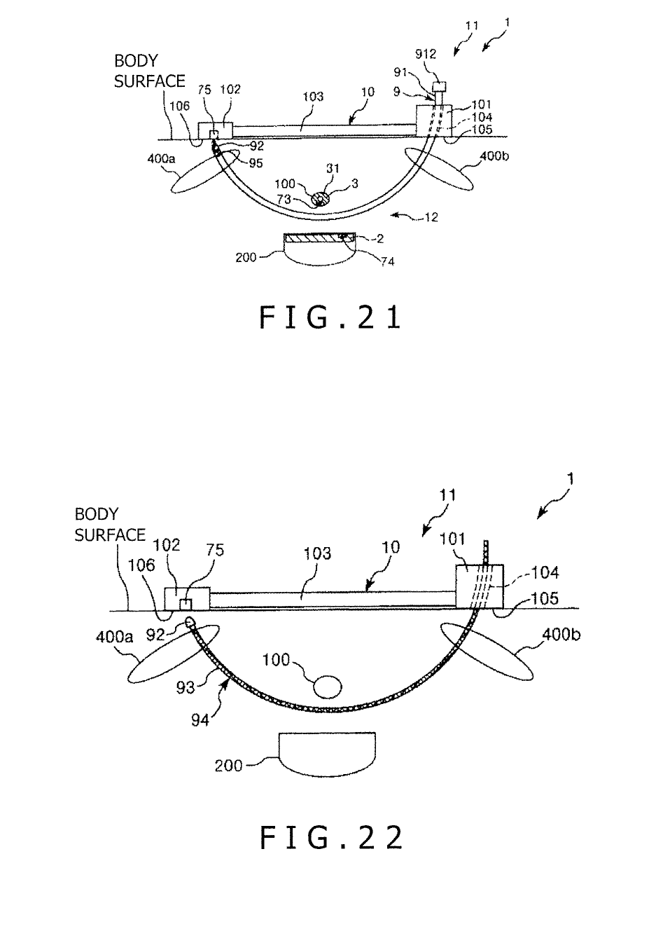

Furthermore, as illustrated in FIG. 21, when the separated distance Lw between the distal portion of the puncture needle assembly 9 and the guide portion 102 is detected and the separated distance Lw becomes a target value Lwp, predetermined information is displayed on the display unit 133 and the buzzer 134 is sounded. Thus, the operator ends the rotary movement operation of the puncture needle assembly 9, which can help prevent the puncture needle assembly 9 from penetrating the body surface on the side of the guide portion 102 and forming a non-through-hole as the puncture hole for implanting the implant 93 in living body tissues.

Next, as illustrated in FIG. 22, only the outer tube 91 is pulled from the puncture needle assembly 9 in the opposite direction of the aforementioned direction and the outer tube 91 is withdrawn. The puncture needle assembly 9 thereby becomes the decomposed state and only the inside structural body 94 is indwelled in living body tissues. Furthermore, by engagement of the step portion 922 of the needle body 92 with living body tissues, this inside structural body 94 is prevented from involuntarily dropping off from living body tissues.

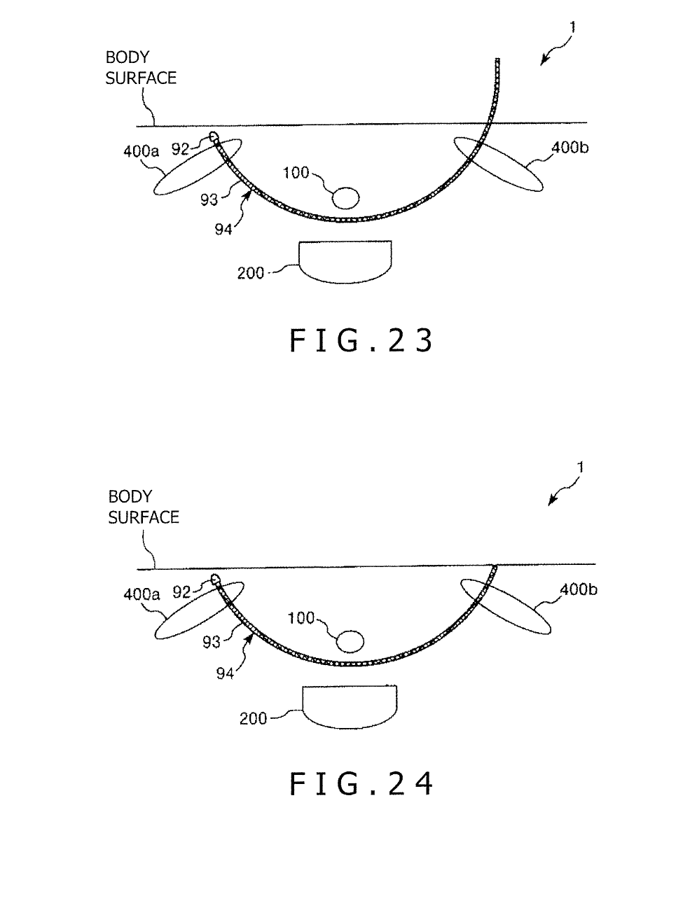

Next, as illustrated in FIG. 23, the support member 10 is removed from the body surface of the patient and the second apparatus 12 is detached from the patient.

Next, as illustrated in FIG. 24, the length of the implant 93 of the inside structural body 94 is adjusted to such a degree as to be able to support the urethra 100 from the lower side in FIG. 24, and the portion of the implant 93 on the opposite side to the needle body 92 is fixed to the body surface. Thereafter, the unnecessary portion of the implant 93 is cut off and predetermined suture and so forth are performed to end the procedure.

According to this puncture apparatus 1, the same effects as those of the aforementioned first embodiment can be achieved.

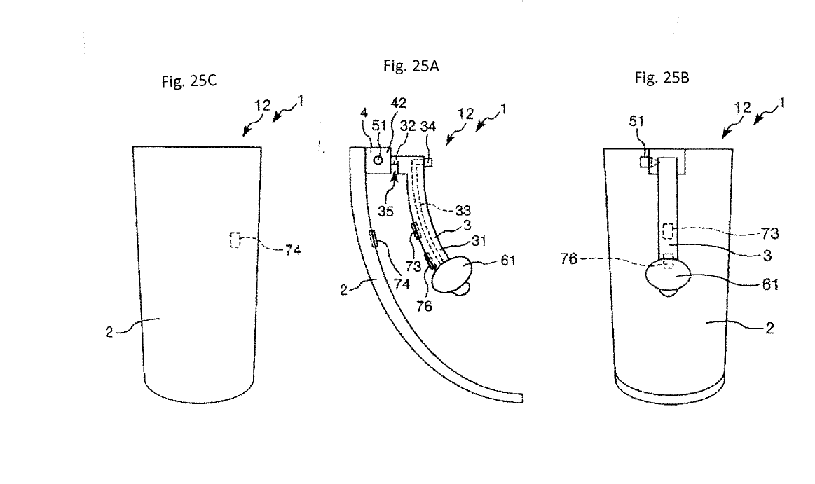

In accordance with an exemplary embodiment, in the following, description will be so made that the lower side is defined as the “distal end” and the upper side is defined as the “proximal end” along the longitudinal direction of a vaginal-insertion portion and a urethral-insertion portion in FIGS. 25A-25C.

In the following, the third exemplary embodiment will be described mainly about differences from the aforementioned second embodiment and description of similar matters will be omitted.

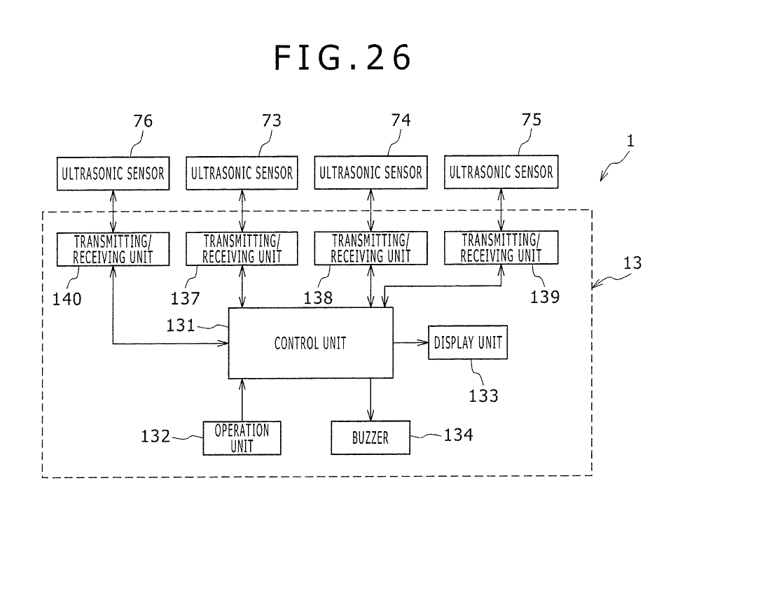

As illustrated in FIGS. 25A-25C and 26, in puncture apparatus 1 of the third exemplary embodiment, an ultrasonic sensor 76 is further disposed on the surface of the middle portion of the urethral-insertion portion 31 on the side of the vaginal-insertion portion 2. This ultrasonic sensor 76 transmits ultrasound toward the side of the vaginal-insertion portion 2. This ultrasonic sensor 76 is disposed on the side of the balloon 61 relative to the ultrasonic sensor 74, for example, disposed near the balloon 61.

Furthermore, the apparatus main body 13 further has a transmitting/receiving unit 140 that performs signal transmission and reception to and from the ultrasonic sensor 76.

In accordance with an exemplary embodiment, detection means that detects information relating to the positional relationship between the distal portion of the puncture needle assembly 9 and two different regions of the urethral-insertion portion 31 in the longitudinal direction, the vaginal-insertion portion 2, and the guide portion 102 and information relating to the positional relationship between the urethral-insertion portion 31 and the vaginal-insertion portion 2 is configured by the control unit 131, the transmitting/receiving units 137 to 140, and the ultrasonic sensors 73 to 76.

In this puncture apparatus 1, the control unit 131 transmits ultrasound by the ultrasonic sensor 76. This ultrasound reflects at the reflective portion 95 provided on the puncture needle assembly 9 and is received by the ultrasonic sensor 76. Then, the control unit 131 measures the time from the transmission of the ultrasound by the ultrasonic sensor 76 to the reception and obtains separated distance Lub between the distal portion of the puncture needle assembly 9 and the urethral-insertion portion 31.

The control unit 131 can compare the separated distance Lub detected by using this ultrasonic sensor 76 with separated distance Lu between the distal portion of the puncture needle assembly 9 and the urethral-insertion portion 31, detected by using the ultrasonic sensor 73, and determines whether or not the distal portion of the puncture needle assembly 9 is too close to the bladder.

If the separated distance Lub is smaller than the separated distance Lu, the distal portion of the puncture needle assembly 9 is too close to the bladder and there is a possibility that the bladder is punctured by the puncture needle assembly 9. Thus, a predetermined alarm can be made by each of the display unit 133 and the buzzer 134. Specifically, for example, predetermined alarm information can be displayed on the display unit 133 and the buzzer 134 can be sounded, which can allow the operator to understand the situation and help prevent the puncture needle assembly 9 from puncturing the bladder.

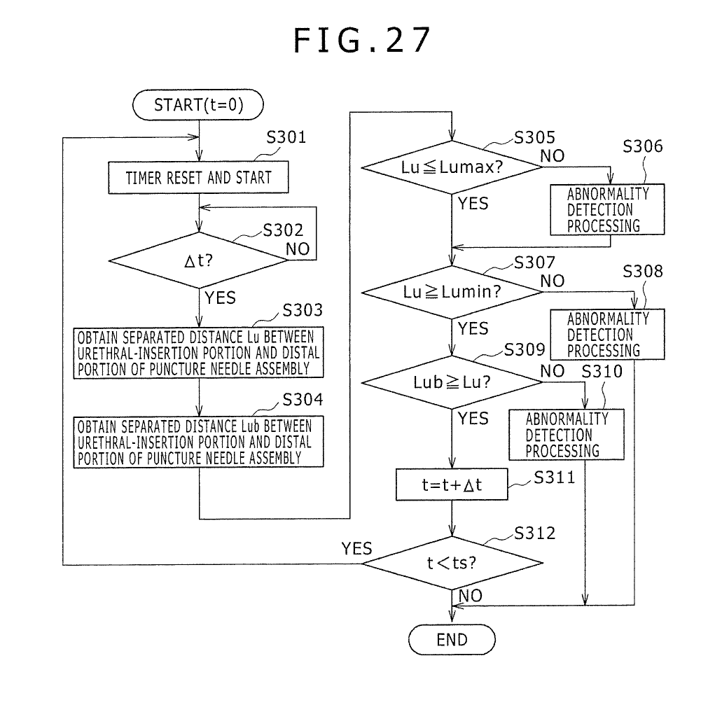

Next, the control operation of the control unit 131 of the puncture apparatus 1 in the case of detecting the separated distances Lu and Lub between the distal portion of the puncture needle assembly 9 and the urethral-insertion portion 31 will be described based on the flowchart illustrated in FIG. 27.

First, measurement time is during which measurement is performed, measurement interval Δt, and maximum value Lumax and minimum value Lumin of the allowable range of the separated distance Lu are set in advance. In accordance with an exemplary embodiment, the measurement time ts, measurement interval Δt, maximum value Lumax, and minimum value Lumin can each be arbitrarily set by the operation unit 132. Then, when a start button, not illustrated, of the operation unit 132 is pressed, the control unit 131 carries out control described below.

As illustrated in FIG. 27, time t indicating the elapsed time from the start of this control is set to 0 and the control unit 131 first resets and starts a timer not illustrated to start measurement of the measurement interval Δt (step S301).

Subsequently, the control unit 131 determines whether the time t has reached the measurement interval Δt (step S302). If the time t has reached the measurement interval Δt, the control unit 131 obtains the separated distance Lu (step S303) and obtains the separated distance Lub (step S304).

In the step S303, the control unit 131 carries out transmission and reception of ultrasound by the ultrasonic sensor 73 and obtains the time between the timing of the transmission of the ultrasound and the timing of the reception. Based on the time, the control unit 131 calculates the separated distance Lu. Furthermore, in the step S304, the control unit 131 carries out transmission and reception of ultrasound by the ultrasonic sensor 76 and obtains the time between the timing of the transmission of the ultrasound and the timing of the reception. Based on the time, the control unit 131 calculates the separated distance Lub.

Subsequently, the control unit 131 determines whether or not the obtained separated distance Lu is equal to or smaller than the maximum value Lumax of the allowable range (step S305). If the separated distance Lu is larger than the maximum value Lumax of the allowable range, the control unit 131 executes abnormality detection processing (step S306).

In this abnormality detection processing (step S306), the control unit 131 displays alarm information on the display unit 133 and sounds the buzzer 134 for example. As the alarm information, for example, “the needle exists at a position remote from the urethra,” “please check whether the urethral-insertion portion is correctly set,” can be used.

The operator can know the existence of some kind of abnormality due to the sounding of the buzzer 134 and can understand the contents of the abnormality, coping methods, and so forth according to the contents of the displaying on the display unit 133.

Furthermore, if the separated distance Lu is equal to or smaller than the maximum value Lumax of the allowable range in the step S305, the control unit 131 determines whether or not the obtained separated distance Lu is equal to or larger than the minimum value Lumin of the allowable range (step S307). If the separated distance Lu is smaller than the minimum value Lumin of the allowable range, the control unit 131 executes the abnormality detection processing (step S308) to end this program.

In this abnormality detection processing, the control unit 131 displays alarm information on the display unit 133 and sounds the buzzer 134 for example. As the alarm information, for example, “the needle exists at a position close to the urethra,” “please temporarily stop puncture operation of the needle and check the state of the urethra,” can be used.

The operator can know the existence of some kind of abnormality due to the sounding of the buzzer 134 and can understand the contents of the abnormality, coping methods, and so forth according to the contents of the displaying on the display unit 133.

Furthermore, if the obtained separated distance Lu is equal to or larger than the minimum value Lumin of the allowable range in the step S307, the control unit 131 determines whether or not the separated distance Lub is equal to or larger than the separated distance Lu (step S309). If the separated distance Lub is smaller than the separated distance Lu, the control unit 131 executes the abnormality detection processing (step S310).

In this abnormality detection processing, the control unit 131 displays alarm information on the display unit 133 and sounds the buzzer 134 for example. As the alarm information, for example, “the needle exists at a position close to the bladder,” “please temporarily stop puncture operation of the needle and check the state of the bladder,” can be used.

The operator can know the existence of some kind of abnormality due to the sounding of the buzzer 134 and can understand the contents of the abnormality, coping methods, and so forth according to the contents of the displaying on the display unit 133.

Furthermore, if the separated distance Lub is equal to or larger than the separated distance Lu in the step S309, the control unit 131 adds the measurement interval Δt to the time t (step S311).

Next, the control unit 131 determines whether the time t is smaller than the measurement time ts (step S312). If the time t is smaller than the measurement time ts, the control unit 131 returns to the step S301 and carries out the step S301 and the subsequent steps again. That is, the control unit 131 carries out the respective steps of the step S303 and the subsequent steps at a time interval of Δt.

Furthermore, in accordance with an exemplary embodiment, if the time t is equal to or larger than the measurement time ts in the step S312, the control unit 131 ends this program.

According to this puncture apparatus 1, the same effects as those of the aforementioned second exemplary embodiment are achieved.

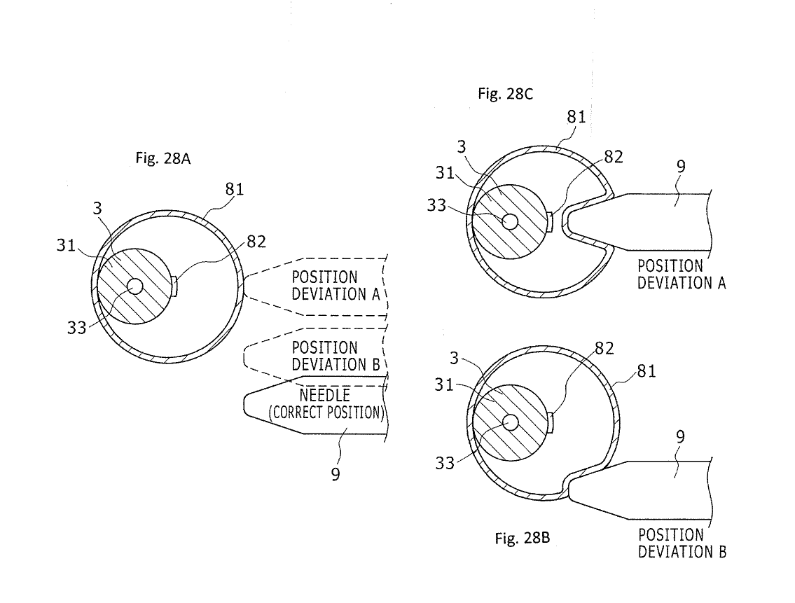

FIG. 28 is a partial sectional view illustrating a fourth exemplary embodiment of the puncture apparatus of the present disclosure. In accordance with an exemplary embodiment, a puncture needle assembly is schematically illustrated in FIG. 28.

In accordance with an exemplary embodiment, in the following, description will be so made that the left side is defined as the “distal end” and the right side is defined as the “proximal end” along the longitudinal direction of the puncture needle assembly in FIG. 28.

In the following, the fourth embodiment will be described mainly about differences from the aforementioned first embodiment and description of similar matters will be omitted.

As illustrated in FIGS. 28A-28C, the puncture apparatus 1 of the fourth exemplary embodiment has, instead of the ultrasonic sensor and the reflective portion, a balloon 81 provided for the urethral-insertion portion 31 and a pressure sensor 82 that is provided in the balloon 81 of the urethral-insertion portion 31 and detects the pressure in the balloon 81 as the detection means that detects information relating to the positional relationship between the distal portion of the puncture needle assembly 9 and the urethral-insertion portion 31. In accordance with an exemplary embodiment, the configuration of the balloon 81 and so forth is similar to that of the aforementioned balloon 61 and so forth and therefore description thereof is omitted. Furthermore, the aforementioned balloon 61 may be substituted as the balloon 81.

A detection result of the pressure sensor 82, for example, a signal indicating the pressure in the balloon 81 detected by the pressure sensor 82, is input to the control unit 131 and the control unit 131 carries out respective kinds of control based on the detection result of the pressure sensor 82.

For example, as illustrated in FIG. 28A, when the distal portion of the puncture needle assembly 9 is located at a proper position with respect to the urethral-insertion portion 31, the distal portion of the puncture needle assembly 9 is separate from the balloon 81 and is not deforming the balloon 81. Thus, the detection result of the pressure sensor 82 is a result corresponding to this. In this case, the distal portion of the puncture needle assembly 9, for example, the needle tip 921, passes between the urethral-insertion portion 31 and the vaginal-insertion portion 2 and can be prevented from puncturing the urethra and the vagina.

Furthermore, in each of a case in which the distal portion of the puncture needle assembly 9 is slightly too close to the urethral-insertion portion 31 as illustrated in FIGS. 28A and 28B (position deviation B) and a case in which the distal portion of the puncture needle assembly 9 is too close to the urethral-insertion portion 31 as illustrated in FIGS. 28A and 28C (position deviation A), there is a possibility that the urethra is punctured by the puncture needle assembly 9. In the case of the position deviation B, a high pressure compared with the case in which the distal portion of the puncture needle assembly 9 is separate from the balloon 81 and is not deforming the balloon 81 is detected by the pressure sensor 82. In addition, in the case of the position deviation A, a high pressure compared with the case of the position deviation B is detected by the pressure sensor 82. Based on the detection result of the pressure sensor 82, the control unit 131 makes a predetermined alarm by each of the display unit 133 and the buzzer 134 as described above. Specifically, as described above, predetermined alarm information is displayed on the display unit 133 and the buzzer 134 is sounded, which can allow the operator to understand the situation and prevent the puncture needle assembly 9 from puncturing the urethra. In accordance with an exemplary embodiment, the configuration may be so made that pieces of alarm information different from each other between the position deviation B and the position deviation A are displayed on the display unit 133.

According to this puncture apparatus 1, the same effects as those of the aforementioned first exemplary embodiment are achieved.

Although the puncture apparatus of the present disclosure is described above about the embodiments illustrated in the diagrams, the present disclosure is not limited thereto and each portion configuring the puncture apparatus can be replaced by an arbitrary configuration capable of exerting a similar function. Furthermore, an arbitrary constituent object may be added.

Moreover, the puncture apparatus of the present disclosure may be apparatus obtained by combining arbitrary two or more configurations (characteristics) of the respective embodiments.

In accordance with an exemplary embodiment, although the respective distances themselves are obtained as the pieces of information relating to the respective positional relationships in the embodiments, the present disclosure is not limited thereto and the configuration may be so made that for example, values proportional to the respective distances or the like are obtained as the pieces of information relating to the respective positional relationships.

Furthermore, in the present disclosure, the configuration may be so made that ultrasonic image data is created based on a signal transmitted from the ultrasonic sensor (ultrasonic oscillator) and an ultrasonic image is displayed.

Moreover, in the embodiments, the configuration is so made that the pieces of information relating to the respective positional relationships are detected by carrying out transmission and reception of ultrasound by the ultrasonic sensor or detecting the pressure by the pressure sensor. However, the present disclosure is not limited thereto and the configuration may be so made that the pieces of information relating to the respective positional relationships are detected optically or magnetically for example.

In the case of optically detecting the pieces of information relating to the respective positional relationships, for example a light emitting element is provided on the puncture needle assembly (puncture needle) and a light receiving element is provided on each of the urethral-insertion portion, the vaginal-insertion portion, and the guide portion of the support member. In accordance with an exemplary embodiment it is preferable to use, as the light emitting element, an element that emits light whose transmittance to living body tissues is comparatively high, specifically for example, near-infrared light.

In the case of magnetically detecting the pieces of information relating to the respective positional relationships, for example a permanent magnet is provided on the puncture needle assembly (puncture needle) and a magnetic sensor is provided on each of the urethral-insertion portion, the vaginal-insertion portion, and the guide portion of the support member.

Puncture apparatus of the present disclosure is characterized by including an insertion portion that is insertable into a living body, a puncture needle that punctures living body tissues near the insertion portion in a state in which the insertion portion is inserted into the living body, and detection means that detects information relating to a positional relationship between a distal portion of the puncture needle and the insertion portion.

According to the present disclosure, the region that must not be punctured can be prevented from being punctured when living body tissues are punctured by the puncture needle. In addition, the burden on the patient is light and the safety of the patient is high. Furthermore, the safety of the operator is also high.

For example, when the puncture apparatus of the present disclosure is used for treatment of urinary incontinence of a woman, the insertion portion of the puncture apparatus is inserted into a urethra and the puncture needle is rotationally moved to puncture the living body by the puncture needle. At this time, information relating to the positional relationship between the distal portion of the puncture needle and the insertion portion can be detected and thus puncture of the urethra by the puncture needle can be prevented.

Furthermore, in implanting an implant for treatment of urinary incontinence, incision of the vaginal wall is unnecessary and the implant can be implanted by a less-invasive procedure. Moreover, the operator can also prevent damage to a fingertip.

Therefore, the puncture apparatus of the present disclosure has industrial applicability.

Having described, by way of example, embodiments of the puncture apparatus and method, it is to be understood that the invention here is not limited to those precise embodiments and that various changes and modifications could be effected therein by one skilled in the art without departing from the spirit or scope of the invention as defined in the claims.

What is claimed is:

1. A puncture apparatus comprising:
an insertion portion that is insertable into a living body;
a puncture needle that punctures living body tissues near the insertion portion in a state in which the insertion portion is inserted into the living body; and
detection means that detects information relating to a positional relationship between a distal portion of the puncture needle and the insertion portion.

2. The puncture apparatus according to claim 1,
wherein the insertion portion is at least one of a urethral-insertion portion that is inserted into a urethra and has an elongated shape and a vaginal-insertion portion that is inserted into a vagina and has an elongated shape.

3. The puncture apparatus according to claim 1,
wherein the insertion portion is a urethral-insertion portion that is inserted into a urethra and has an elongated shape; and
the puncture apparatus has a restriction unit that is provided on the urethral-insertion portion and restricts a position of the urethral-insertion portion in a longitudinal direction in the urethra.

4. The puncture apparatus according to claim 1,
wherein the detection means has a function to detect information relating to positional relationships between the distal portion of the puncture needle and at least two different regions of the insertion portion in a longitudinal direction.

5. The puncture apparatus according to claim 1, wherein the insertion portion comprises:
a urethral-insertion portion that is inserted into a urethra and has an elongated shape; and
a vaginal-insertion portion that is inserted into a vagina and has an elongated shape.

6. The puncture apparatus according to claim 5,
wherein the detection means has a function to detect information relating to positional relationships between the distal portion of the puncture needle and at least two different regions of the urethral-insertion portion and/or the vaginal-insertion portion in a longitudinal direction.

7. The puncture apparatus according to claim 5,
wherein the detection means has a function to detect information relating to a positional relationship between the urethral-insertion portion and the vaginal-insertion portion.

8. The puncture apparatus according to claim 7, comprising:
informing means that informs information based on a detection result of the detection means.

9. The puncture apparatus according to claim 8, comprising:
determination means that determines whether a separated distance between the urethral-insertion portion and the vaginal-insertion portion is equal to or larger than a minimum value of an allowable range; and
wherein an alarm is made by the informing means if it is determined by the determination means that the separated distance between the urethral-insertion portion and the vaginal-insertion portion is smaller than the minimum value of the allowable range.

10. The puncture apparatus according to claim 8, comprising:
determination means that determines whether a separated distance between the urethral-insertion portion and the vaginal-insertion portion is equal to or smaller than a maximum value of an allowable range; and
wherein an alarm is made by the informing means if it is determined by the determination means that the separated distance between the one and the other is larger than the maximum value of the allowable range.

11. The puncture apparatus according to claim 1, comprising:
informing means that informs information based on a detection result of the detection means.

12. The puncture apparatus according to claim 1, comprising:
informing means that informs information based on a detection result of the detection means;
determination means that determines whether a separated distance between the distal portion of the puncture needle and the insertion portion is equal to or larger than a minimum value of an allowable range; and
wherein an alarm is made by the informing means if it is determined by the determination means that the separated distance between the distal portion of the puncture needle and the insertion portion is smaller than the minimum value of the allowable range.

13. The puncture apparatus according to claim 1, comprising:
informing means that informs information based on a detection result of the detection means;
determination means that determines whether separated distance between the distal portion of the puncture needle and the insertion portion is equal to or smaller than a maximum value of an allowable range; and
wherein an alarm is made by the informing means if it is determined by the determination means that the separated distance between the distal portion of the puncture needle and the insertion portion is larger than the maximum value of the allowable range.

14. The puncture apparatus according to claim 4, comprising:
informing means that informs information based on a detection result of the detection means;
comparison means that compares one of separated distances between the distal portion of the puncture needle and the at least two different regions of the insertion portion in the longitudinal direction with the other; and
wherein the informing means informs information based on a comparison result of the comparison means.

15. The puncture apparatus according to claim 1,
wherein the puncture needle is curved into a shape of a circular arc; and
the puncture apparatus has a support portion that supports the puncture needle rotationally movably around a center axis of the circular arc, and a guide portion that is provided at a different position from the support portion and is located in a direction toward which a needle tip of the puncture needle moves when the puncture needle rotationally moves.

16. The puncture apparatus according to claim 1,
wherein the puncture needle is formed of a puncture needle assembly that includes an outer tube that is formed of a tubular body curved into a shape of a circular arc and has a one-end opening obtained by opening of one end of the outer tube and a needle body that is freely detachably mounted at the one-end opening and has a rounded, non-sharp needle tip to puncture living body tissues, the puncture needle assembly being capable of taking an assembled state in which the puncture needle assembly is assembled by mounting the needle body at the one-end opening; and
the puncture apparatus has a support portion that supports the puncture needle assembly in the assembled state rotationally movably around a center axis of the circular arc of the outer tube, and a guide portion that is provided at a different position from the support portion and is located in a direction toward which the needle tip moves when the puncture needle assembly rotationally moves.

17. A puncture apparatus comprising:
a puncture needle that punctures living body tissues near the insertion portion in a state in which the insertion portion is inserted into the living body, the puncture needle having at least one ultrasonic sensor on a distal portion of the puncture needle;
an insertion portion that is insertable into a living body, the insertion portion comprising at least one of a urethral-insertion portion that is inserted into a urethra and has an elongated shape and a vaginal-insertion portion that is inserted into a vagina and has an elongated shape, the insertion portion having at least one reflective portion that reflects ultrasound; and
detection means that detects information relating to a positional relationship between the distal portion of the puncture needle and the insertion portion based on the at least one ultrasonic sensor on the puncture needle and the at least one reflective portion on the insertion portion.

18. A method of preventing a region of a living body from being punctured, comprising:
inserting an insertion portion into a living body, the insertion portion comprising at least one of a urethral-insertion portion that is inserted into a urethra and has an elongated shape and a vaginal-insertion portion that is inserted into a vagina and has an elongated shape;
inserting a puncture needle that punctures living body tissues near the insertion portion in a state in which the insertion portion is inserted into the living body;
detecting a positional relationship between a distal portion of the puncture needle and the insertion portion; and
informing an operator of the positional relationship between the distal portion of the puncture needle and the insertion portion using an informing means.

19. The method according to claim 18,
wherein the insertion portion comprises the urethral-insertion portion and the vaginal-insertion portion;
determining whether a separated distance between the urethral-insertion portion and the vaginal-insertion portion is equal to or larger than a minimum value of an allowable range, and wherein an alarm is made by the informing means if it is determined by the determination means that the separated distance between the urethral-insertion portion and the vaginal-insertion portion is smaller than the minimum value of the allowable range; or
determining whether the separated distance between the urethral-insertion portion and the vaginal-insertion portion is equal to or smaller than a maximum value of an allowable range, and wherein the alarm is made by the informing means if it is determined by the determination means that the separated distance between the one and the other is larger than the maximum value of the allowable range.

20. The method according to claim 18, comprising:
determining whether a separated distance between the distal portion of the puncture needle and the insertion portion is equal to or larger than a minimum value of an allowable range, and wherein an alarm is made by the informing means if it is determined by the determination means that the separated distance between the distal portion of the puncture needle and the insertion portion is smaller than the minimum value of the allowable range, or
determining whether separated distance between the distal portion of the puncture needle and the insertion portion is equal to or smaller than a maximum value of an allowable range, and wherein the alarm is made by the informing means if it is determined by the determination means that the separated distance between the distal portion of the puncture needle and the insertion portion is larger than the maximum value of the allowable range.
